SEQUENCE LISTING (6 sequences)

SEQ ID NO: 1 - DNA, 1758 bp, Homo sapiens
  misc_feature (1)..(1758): cDNA encoding HSA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 2 - protein, 585 aa, Homo sapiens [sequence omitted]

SEQ ID NO: 3 - protein, 214 aa, Artificial sequence
  Bevacizumab light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEEDSKDSTYSLSSTLTLSKADYEKHKVYACEVTHQGLSSPVTKSFNRGEC

SEQ ID NO: 4 - protein, 453 aa, Artificial sequence
  Bevacizumab heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 213 aa, Artificial sequence
  Rituximab light chain chimeric
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 451 aa, Artificial Sequence
  Rituximab heavy chain chimeric
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2016/0250329 A1
(43) Pub. Date: Sep. 1, 2016

(54) ANTIBODY COMPOSITION

(71) Applicant: ALBUMEDIX A/S, Kgs. Lingby (DK)

(72) Inventors: Jens Thostrup Bukrinski, Bagsvaerd (DK); Anne Marie Scharff-Poulsen, Bagsvaerd (DK); Corinne Diane Eenschooten, Bagsvaerd (DK); Mette-Marie List Jensen, Bagsvaerd (DK); Mette Larsen, Bagsvaerd (DK)

(21) Appl. No.: 15/032,870

(22) PCT Filed: Oct. 29, 2014
(86) PCT No.: PCT/EP2014/073261

§ 371 Date: Apr. 28, 2016

(30) Foreign Application Priority Data
Oct. 29, 2013 (EP) 13190750.3
May 2, 2014 (EP) 14166865.7

(51) Int. Cl. A61K 39/395; C07K 16/28; C07K 16/22

(52) U.S. Cl. CPC A61K 39/39591; C07K 16/22; A61K 39/3955; C07K 16/2887; C07K 2317/94; C07K 2317/24; A61K 2039/545

(57) ABSTRACT

The invention provides a method for stabilizing antibody at high concentrations using albumin; use of albumin to stabilize high concentration antibody compositions; a stable high concentration antibody composition and uses thereof. The invention also provides a method to control or reduce viscosity and/or injection force of an antibody composition.

### REFERENCE TO A SEQUENCE LISTING

This application contains a Sequence Listing in computer readable form, which is incorporated herein by reference.

### BACKGROUND OF THE INVENTION

1. Field of the Invention

The invention provides a method for stabilizing antibody at high concentrations using albumin; use of albumin to stabilize high concentration antibody compositions; a stable high concentration antibody composition and uses thereof.

2. Description of the Related Art

Antibodies are useful in biology and medicine (Waldmann (2003), Nature Medicine 9(3): 269-277) such as for passive immunotherapy. By 2011, the US Food and Drug Administration (FDA) had approved 26 monoclonal antibody drugs for clinical use against cancer (Vazquez-Rey (2011) Biotechnology and Bioengineering, 108(7): 1494-1508). Many more antibody-based drugs are the subject of ongoing clinical trials.

Antibody treatments often require a high dose, such as several milligrams of antibody per kilogram of patient body mass. To date, it has not been possible to provide a high concentration antibody composition having an acceptable level of stability. Aggregation of highly concentrated antibody compositions may result in production of aggregates such as dimers, multimers, polymers and/or particulates of antibodies which may cause adverse immunogenic reactions, may affect antibody activity, may affect dosing precision, and may affect pharmacokinetic properties of the antibody. Overall, antibody aggregation may result in compromised safety and efficacy.

The challenge of stabilizing antibodies has been addressed by adding sugars, polyols, solvents and detergents (e.g. polysorbate 20 and polysorbate 80) to an antibody composition (Vazquez-Rey (2011), op. cit.). For example, WO98/56418 describes stabilization of relatively low concentration antibody compositions using acetate buffer, surfactant and polyol. Other strategies include mutating the amino acid sequence of an antibody to alter the charge of the complementarity determining region (‘CDR’) (Bethea et al (2012), Protein Engineering, Design & Selection, 1-7). However, it has not been possible to provide antibodies at sufficiently high concentrations to allow subcutaneous administration due to problems associated with protein instability (Shire et al (2004) Journal of Pharmaceutical Science 93, 1390-1402). The re-publication of Shire et al in 2010 (‘Challenges in the Development of High Protein Concentration Formulations’, Chapter 9, Current Trends in Monoclonal Antibody Development and Manufacturing, Biotechnology: Pharmaceutical Aspects, American Association of Pharmaceutical Scientists) shows how difficult this challenge is to overcome.

Consequently, current antibody compositions are provided at undesirably low concentrations and therefore each dose of drug has a large volume which results in the need for multiple injections or intravenous (IV) administration. For example, typical antibody compositions used in medicine are provided at a concentration of around 10 to 100 mg/mL. Compositions containing antibody concentrations at the higher end of this range are not particularly stable. Furthermore, prior to administration to a patient, the antibody compositions are diluted to lower concentrations such as 1 to 20 mg/mL.

The maximum allowable volume that can be delivered subcutaneously is typically about 1.5 mL due to the pain associated with receiving larger volumes. Higher volumes require intravenous administration. Delivery of large volumes of a drug to a patient can require considerable time, e.g. from 30 to 90 minutes per dose. Consequently, administration of the drug requires the patient to be present in a hospital (‘in patient’) to receive treatment. This is inconvenient to the patient and may lead to non-compliance with a recommended treatment plan. Treating a patient in a hospital, or other medical facility, is expensive for healthcare providers. Storage and pharmaceutical compounding of large volumes of drugs is expensive.

It would be advantageous to patients and to healthcare providers if the dose volume of an antibody composition could be reduced to a volume that allows administration in a more convenient medical facility (e.g. a community healthcare practice for administration by a general practice doctor, nurse or healthcare assistant), by a patient's friend or family member or by the patient himself. For example, subcutaneous administration would be desirable. It is desirable to provide a high concentration albumin composition which overcomes one or more (several) of the problems described above.

### SUMMARY OF THE INVENTION

Therefore, the invention provides a method for stabilizing antibody at high concentrations using albumin.

The invention also provides use of albumin to stabilize a high concentration antibody composition.

The invention also provides a stable high concentration antibody composition.

The invention also provides uses of a stable high concentration antibody composition, for example in treatment or prevention of disease or a medical condition.

The invention also provides a container holding a stable high concentration albumin composition.

### DEFINITIONS

Albumin: The term ‘albumin’ means a protein having the same and/or very similar three dimensional (tertiary) structure as human serum albumin (‘HSA’, SEQ ID NO: 2) or one or more HSA domain and has similar properties to HSA or to the relevant domain or domains. Similar three dimensional structures are, for example, the structures of HSA. Some of the major properties of albumin are i) its ability to regulate plasma volume (oncotic activity), ii) a long plasma half-life of around 19 days±5 days, iii) binding to FcRn, iv) ligand-binding, e.g. binding of endogenous molecules such as acidic, lipophilic compounds including bilirubin, fatty acids, hemin and thyroxine (see also Table 1 of Kragh-Hansen et al, 2002, Biol. Pharm. Bull. 25, 695, hereby incorporated by reference), v) binding of small organic compounds with acidic or electronegative features e.g. drugs such as warfarin, diazepam, ibuprofen and paclitaxel (see also Table 1 of Kragh-Hansen et al, 2002, Biol. Pharm. Bull. 25, 695, hereby incorporated by reference). Not all of these properties need to be fulfilled in order to characterize a protein or fragment as an albumin. If a fragment, for example, does not comprise a domain responsible for binding of certain ligands or organic compounds the variant of such a fragment will not be expected to have these properties either. HSA (SEQ ID NO: 2) may be encoded by a nucleotide sequence such as SEQ ID NO: 1.

Antibody: The term ‘antibody’ or ‘antibody molecule’ includes whole antibodies (e.g. Immunoglobulin G (IgG), Immunoglobulin A (IgA), Immunogolbulin E (IgE), Immunoglobulin M (IgM), or Immunoglobulin D (IgD)), and antibody fragments such as Fab, F(ab′)2, Fab3, scFv, Fv, dsFv, ds-scFv, Fd, dAbs, TandAbs, minibodies, diabodies, tribodies, tetrabodies, vH domain, vL domain, v_{H}H domain, Nanobodies, IgNAR variable single domain (v-NAR domain), fragments thereof, and multimers thereof and bispecific antibody fragments. Antibodies include monoclonal antibodies (‘mAbs’), polyclonal antibodies, and chimeric antibodies.

Buffer: means a solution that resists changes in pH by the action of its acid-base conjugate components. Examples of buffers that control pH include acetate (e.g. sodium acetate), succinate (e.g. sodium succinate), gluconate, histidine, citrate and other organic acid buffers. For a freeze-thaw stable formulation, a phosphate buffer is not preferred.

CDR: refers to a complementarity determining region within antibody variable sequences. There are usually three CDRs in each of the variable regions of the heavy chain and the light chain, which are designated CDR1, CDR2 and CDR3 for each of the variable regions. The invention is not limited to antibodies, antibody fragments, or antibody fusions with only three CDRs in each variable region.

Domain: with regard to the immunoglobulins, ‘domain’ is a folded protein structure which retains its tertiary structure independent of the rest of the protein. Generally, domains are responsible for discrete functional properties of proteins, and in many cases may be added, removed or transferred to other proteins without loss of function of the remainder of the protein and/or of the domain. A ‘single antibody variable domain’ is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

Fragment: The term ‘fragment’ means from 20, 30, 40, 50, 60, 70, 80, or 90 to 30, 40, 50, 60, 70, 80, 90, 95, 96, 97, 98, 99 or 99.9% of the length of the peptide from which the fragment is derived. It is preferred that the fragment has at least 50%, more preferably 60, 70, 80, 90, 05, 99 or 100% of the functional activity of the peptide from which it is derived.

The term ‘antibody fragment’ refers to antibody fragments of biological relevance, e.g. fragments which can contribute to or enable antigen binding, e.g. from part or all of the antigen binding site, or can contribute to the inhibition or reduction in function of the antigen or can contribute to the prevention of the antigen interacting with its natural ligands. Preferred fragments thus comprise a heavy chain variable region (vH domain) and/or a light chain variable region (vL domain) of the antibodies of the invention. Other preferred fragments comprise one or more of the heavy chain complementarity determining regions (CDRs) of the antibodies of the invention (or of the vH domains of the invention), or one or more of the light chain CDRs of the antibodies of the invention (or of the vL domains of the invention).

The term ‘albumin fragment’ includes albumin having an amino acid sequence length of at least 60, 70, 80, 90, 95, 96, 97, 99, or 99.5% of that of SEQ ID NO: 2 (585 amino acids). A fragment may comprise two or more domains of albumin such as Domain I and Domain II, Domain I and Domain III or Domain II and Domain III.

When used in the context of a nucleic acid molecule, the term ‘fragment’ includes a nucleic acid molecule encoding a fragment as described herein.

Immunoglobulin: The term ‘immunoglobulin’ refers to a family of polypeptides which retain the immunoglobulin fold characteristic of antibody molecules, which contains two beta sheets and, usually, a conserved disulfide bond.

Isotonic: The term ‘isotonic’ means having essentially the same osmotic pressure as human blood. Isotonic compositions typically have an osmotic pressure from about 250 to 350 mOsm. A vapour pressure or ice-freezing type osmometer may be used to measure isotonicity.

Mammal: includes humans, domestic and farm animals (e.g. cows, sheep, pigs, horses), and zoo, sports (e.g. dogs or horses), or pet animals (e.g. dogs, cats, rabbits). Preferably, the mammal is human.

Monoclonal antibody: means an antibody obtained from a population of substantially homogeneous antibodies.

Non-reducing sugars: include sucrose, trehalose, sorbose, melezitose and raffinose.

Particles or particulates: include the subset of aggregates having a size of at least 1 micrometer, such as from 1 to 100 micrometers, most preferred 2 to 10 micrometers. Size may be equivalent circular diameter “ECD”, i.e. the diameter of the smallest circle that can be drawn around the particle to completely encircle the particle.

Patient: means a subject who may be treated (therapeutically or prophylactically) and may be a mammal such as a human.

Pharmaceutical formulation: preparations in a form which permit the biological activity of the active ingredients to be effective, and which contain no additional components which are toxic to the subjects (e.g. patient) to which the formulation is to be administered.

Pharmaceutically acceptable excipient: can be administered to a patient to provide an effective dose of the active ingredient employed.

Preservative: a compound which reduces microbial, e.g. bacterial and/or fungal action, and can be useful for generation of multi-use formulations. Preservatives include octadecyidimethylbenzyl ammonium chloride, hexamethonium chloride, benzalkonium chloride (a mixture of alkylbenzyldimethylammonium chlorides in which the alkyl groups are long-chain compounds), and benzethonium chloride, aromatic alcohols (e.g. phenol, butyl and benzyl alcohol, alkyl parabens e.g. methyl or propyl paraben, catechol, resorcinol, cyclohexanol, 3-pentanol, and m-cresol).

Prevention (or inhibition): In relation to aggregates e.g. dimers, polymers, particulates or fibrils, the term ‘prevention’ or ‘inhibition’ means hindering of the formation of aggregates. Prevention or inhibition may be complete, e.g. no aggregates are formed. Prevention or inhibition may be partial, e.g. fewer aggregates are formed compared to a reference composition or the aggregates formed may be incomplete or the aggregates may be formed at a slower rate. A ‘reference’ composition may be a composition in which albumin is absent. For example, partial inhibition of aggregation may result in a composition containing at most about 99, 98, 97, 96, 95, 90, 85, 80, 75, 70, 65, 60, 55, 50, 45, 40, 35, 30, 25, 20, 15, 10, 5, 4, 3, 2, 1, 0.1, 0.01% of the aggregates formed in a reference composition. Prevention (or inhibition) or reduction of aggregation may be measured immediately or following exposure of the composition to stress and quantification of aggregates.

Pure: The antibody which is formulated is preferably essentially pure and desirably essentially homogeneous (i.e. free from contaminating proteins etc). ‘Essentially pure’ antibody means a composition comprising at least about 90% by weight of the antibody, based on total weight of the composition, preferably at least about 95% by weight. ‘Essentially homogeneous’ antibody means a composition comprising at least about 99% by weight of antibody, based on total weight of the composition.

Quantitation of aggregates e.g. antibody dimers, multimers, polymers and/or particulates: may be by SE-HPLC, AF4, DLS or MFI.

Reduction: In relation to aggregates e.g. dimers, the term ‘reduction’ means partial or complete removal of existing aggregates.

Sequence Identity: The relatedness between two amino acid sequences is described by the parameter ‘sequence identity’. For purposes of the invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later, more preferably version 5.0.0 or later. The optional parameters used are gap open penalty of 10, gap extension penalty of 0.5, and the EBLOSUM62 (EMBOSS version of BLOSUM62) substitution matrix. The output of Needle labelled ‘longest identity’ (obtained using the—nobrief option) is used as the percent identity and is calculated as follows:

(Identical Residues×100)/(Length of Alignment−Total Number of Gaps in Alignment)

Stable formulation: a formulation in which the antibody contained therein, following exposure to stress for a selected time period (e.g. at least about 1, 2, 3, 4, 5, 6 months or following storage at about 2 to about 8° C. (e.g. about 5° C.) for at least about 1, 2, 3, 4 or 5 years), contains at least 1 percentage point fewer antibody aggregates e.g. dimers, multimers, polymers and/or antibody particulates compared with a reference composition which is identical to the antibody composition with the exception that it does not comprise albumin; or retains its biological activity to within 10%, more preferably to within 9, 8, 7, 6, 5, 4, 3, 2, 1% of the biological activity exhibited at the time of its formulation e.g. as determined in an antibody-antigen binding assay such as enzyme linked immunosorbent assay (ELISA).

Stress: includes exposure to elevated temperature (e.g. about 25° C. or about 40° C.); shear (e.g. stirring or turning for 30 minutes every 24 hours at 30 to 120 revolutions per minute (rpm)); freeze thaw (e.g. freeze to about −20 (minus 20) or about −70 (minus 70° C.); and hydrophobic surfaces.

Therapeutically effective amount of an antibody refers to an amount effective for its use e.g. in prevention or treatment of the disease or medical condition for which the antibody is effective.

### DETAILED DESCRIPTION OF THE INVENTION

A first aspect of the invention provides a composition, especially a liquid composition, of antibody comprising antibody at a concentration of greater than or equal to about 140 mg/mL and albumin at a concentration of greater than or equal to about 10 mg/mL. The liquid composition may be a pharmaceutical formulation such as an aqueous pharmaceutical formulation. Preferably, the composition comprises a therapeutically effective amount of antibody for example sufficient to provide from about 0.1 to about 50 mg/kg of patient body mass (e.g. from about 7.5 to about 3750 mg for a 75 kg patient), such as from about 0.5 to about 25 mg/kg or from about 1 to about 15 mg/kg. The antibody may or may not be subjected to lyophilization prior to inclusion in the composition.

A particularly preferred liquid composition according to the first aspect of the invention is a stable composition such as a stable aqueous pharmaceutical formulation.

The composition (e.g. liquid composition) may comprise antibody at a concentration from about 140, 150, 160, 170, 180, 190, 200, 225, 250, or 275 to about 150, 160, 170, 180, 190, 200, 225, 250, 275, or 300 mg/mL. Preferred concentrations include from about 140 to about 250 mg/mL, from about 140 to about 200 mg/mL and from about 140 to about 175 mg/mL.

For administration using a syringe, it is preferred that the antibody concentration is less than or equal to about 250 mg/mL. For administration using an auto injector, it is preferred that the antibody concentration is less than or equal to about 300 mg/mL

Following exposure to stress, the composition (e.g. liquid composition) may contain at least one percentage point fewer antibody aggregates (e.g. dimers, multimers, polymers and/or particulates) compared with a reference composition which is identical to the antibody composition with the exception that the reference composition does not comprise albumin. For example, a reference composition may comprise antibody in water or in a buffer. More preferably, the composition may contain at least 2, 3, 4, 5, 10, 15, 20, 25, 30, 35, 40, 45, 50, 55, 60, 65, 70, 80, 85% fewer antibody aggregates (e.g. dimers, multimers, polymers and/or particulates) compared with a reference composition which is identical to the antibody composition with the exception that the reference composition does not comprise albumin. The composition may contain at most 99, 98, 97, 96, 95, 90, 85, 80, 75, 70, 65, 60, 55, 50, 45, 40, 35, 30, 25, 20, 15, 10, 5, 4, 3, 2, or 1% of the number of aggregates (e.g. dimers, multimers, polymers and/or particulates) present in a reference composition which is identical to the antibody composition with the exception that the reference composition does not comprise albumin. A composition containing fewer particulates than the reference composition is preferred, particularly particulates in the size range 2 to 10 micrometers. Exposure to stress may be for at least about 1, 2, 3, 4, 5, 6, 7, 28 or 29 days, or at least about 1, 2, 3, 4, 5, 6, 7, 8 or 9 weeks or at least about 1, 2, 3, 4, 5 or 6 months or about 1, 2, 3, 4 or 5 years. More preferably, following exposure to stress, the composition (e.g. liquid composition) contains at least about 2, 3, 4, 5, 6, 7, 8, 9, 10, 15, 20, 25, 30, 35, 40, 45, 50, 55, 60, 65, 70, 75, 80, 85, 90 or 95 percentage points fewer antibody aggregates compared with the reference composition. Following exposure to stress, the composition (e.g. liquid composition) may contain at most 99, 98, 97, 96, 95, 90, 85, 80, 75, 70, 65, 60, 55, 50, 45, 40, 35, 30, 25, 20, 15, 10, 5, 4, 3, 2, or 1% of the number of aggregates present in the reference composition. The stress may be selected from elevated temperature (e.g. about 25° C. or about 40° C.); shear (e.g. incubation at about 25° C. or about 40° C. with stirring or turning for 30 minutes every 24 hours at 30 to 120 revolutions per minute (rpm)); freeze thaw (e.g. 5 to 10 cycles of incubation at about −20° C. (minus twenty) for 1 or more hours, followed by thawing at about 20° C. for 1 hour); and exposure to hydrophobic surfaces (e.g. incubation with Teflon beads, at about 25° C. or about 40° C. with stirring for 30 minutes every 24 hours at 30 to 120 rpm). A preferred stress test is exposure to 40° C. without agitation for about one month, e.g. 28 or 29 days. Another preferred stress test is exposure to 40° C. without agitation for about 2 months, e.g. 9 weeks.

Particulates, or particles, may be, for example, in the size range from about 1 or about 2 to about 100 micrometers, such as from about 1, 2, 3, 4, 5, 10, 20, 25, 30 to about 2, 3, 4, 5, 10, 20, 25, 30, 40, 50, 60, 70, 80, 90 or 100 micrometers, such as from about 20 to about 100 micrometers, from about 4 to about 70 micrometers, from about 10 to about 100 micrometers, from about 25 to about 100 micrometers, most preferably from about 2 to about 20 micrometers.

Antibody aggregates (e.g. antibody dimers, multimers, polymers and/or particulates) may be detected and/or quantified by any suitable method for example size-exclusion high-performance liquid chromatography (SE-HPLC), asymmetrical flow field flow fractionation (AF4), differential light scattering (DLS) or micro flow imaging (MFI).

AF4 is suitable for detecting aggregates in the size range from monomers up to a few hundred nanometers. It allows for separation of albumin (e.g. HSA) and mAb and HSA dimers and mAb monomers. Aggregates can be quantified by determining the relative amount (e.g. percentage) of particles of a particular size (e.g. molecular mass).

DLS is suitable for detecting particles in the size range from about 1 nm to a few hundred nm and provides an estimate of the size of the particles. It additionally determines polydispersity of a solution. DLS is suitable for measuring the mean particle size of a solution such as a bulk solution.

MFI may use a flow microscope to detect particles in the size range from about 1 or about 2 to about 100 micrometers, such as from about 1, 2, 3, 4, 5, 10, 20, 25, 30 to about 2, 3, 4, 5, 10, 20, 25, 30, 40, 50, 60, 70, 80, 90 or 100 micrometers, such as from about 20 to about 100 micrometers, from about 2 to about 10 micrometers, from about 2 to about 25 micrometers, from about 4 to about 70 micrometers, from about 10 to about 100 micrometers, from about 25 to about 100 micrometers. It can be used to detect visible and sub-visible particles.

Preferably, the liquid composition of antibody is visibly clear (e.g. does not show protein participates, crystals or gels) and/or does not sediment during centrifugation (e.g. 30 000×g for 30 minutes). Preferably, following a stress test, the liquid composition remains visibly clear.

The liquid composition may be isotonic, e.g. isotonic relative to patient blood e.g. human blood. A liquid composition being within (plus or minus) 100% of isotonicity such as (plus or minus) 20% of isotonicity is desirable for minimizing pain experience by the patient receiving a drug, such as intravenously.

The composition (e.g. liquid composition) may be stable at a temperature of about 2 to about 8° C. (e.g. about 5° C.) for at least about 1, 2, 3, 4, 5, 6 or 7 days, at least about 1, 2, 3, or 4 weeks, about 1, 2, 3, 4, 5 or 6 months or at least about 1, 2, 3, 4 or 5 years.

The composition (e.g. liquid composition) may be stable following freezing (e.g. to about −20° C. or about −70° C.) and thawing of the formulation, for example a single freeze-thaw cycle or multiple freeze-thaw cycles e.g. 2, 3, 4 or 5 freeze-thaw cycles. Freeze thaw cycles are described herein.

The composition (e.g. liquid composition) may be stable at about 25° C. or 30° C. for at least 1, 2, 3, 4, 5, 6 or 7 days, at least about 1, 2, 3, or 4 weeks, at least about 1, 2, 3, 4, 5 or 6 months or at least about 1, 2, 3, 4 or 5 years. Stability at 25° C. or 30° C. is useful, for example, for distribution logistics and/or to aid patient compliance for example when travelling without access to refrigeration facilities.

With regards the profile of the injection force of the composition (e.g. liquid composition): the composition (e.g. liquid composition) may have an injection force, such as ‘peak force’ (force required to be imparted on the liquid for it to start to flow) of from about 1 to about 30 N, such as from about 1, 2, 3, 4, 5, 6, 7, 8, 9, 10, 11, 12, 13, 14, 15, 16, 17, 18, 19, 20, or 25 to about 2, 3, 4, 5, 6, 7, 8, 9, 10, 11, 12, 13, 14, 15, 16, 17, 18, 19, 20, 25 or 30 N when dispensed using a 30 gauge (G) or, more preferably, a 27 G needle. A peak force of from about 10 to about 15 is preferred. The composition (e.g. liquid composition) may have a ‘time to peak force’ (time taken for the initial application of the force to reaching peak force′) of from about 0 to about 30 seconds, such as from about 1 to about 20 seconds, more preferably from about 1 to about 10 seconds, most preferably from about 1 to about 5 seconds such as at most about 1, 2, 3, 4 or 5 seconds.

It is preferred that the injection force profile is determined using a needle with a gauge from 15 to 34 or higher (i.e. narrower diameter), preferably from 20 G to 34 G or higher. A needle gauge of about 25 to 35, such as about 27 or about 30 is preferred. It is preferred that the injection force profile is determined using a volume of from about 1 to about 5 mL. A volume of about 2 mL is preferred. It is preferred that the injection force profile is determined using an injection rate of about 2 to about 5 mL per minute, preferably about 2 mL per minute.

The antibody may be selected from the group consisting of IgG, IgA, IgM, IgE, and IgD. IgG is particularly preferred. There are four subclasses of IgG: IgG1, IgG2, IgG3 and IgG4; IgG1 is preferred. The antibody may have a molecular weight of from about 125, 130, 135, 140, 145, 150, 155, 160, 165, or 170 kDa to about 130, 135, 140, 145, 150, 155, 160, 165, 170 or 175 kDa. Molecular weights of from about 135 to about 165 are preferred, more preferably from about 145 to about 155 kDa, most preferably about 150 kDa such as about 149 kDa.

Immunoglobulin (Ig) classes are based on small differences in the amino acid sequences in the constant region of the heavy chains. Immunoglobulins within a subclass can have similar heavy chain constant region amino acid sequences, wherein differences are detected by serological means. For example, the IgG subclasses comprise IgG1, IgG2, IgG3, and IgG4, wherein the heavy chain is classified as being a gamma 1 heavy chain, a gamma 2 heavy chain, and so on due to the amino acid differences. The light chain can be of the kappa or lambda type. In another example, the IgA subclasses comprise IgA1 and IgA2, wherein the heavy chain is classified as being an alpha 1 heavy chain or an alpha 2 heavy chain due to the amino acid differences.

Within the present invention, antibodies also include those devoid of light chains, such as those found in camel, llama and other members of the camelidae family, and sometimes referred to as heavy chain antibodies (HcAb). Similarly, ‘antibodies’ include immunoglobulin isotype novel (or new) antigen receptors (IgNARs), which are naturally found in cartilaginous marine animals, for example Wobbegong sharks and nurse sharks, and other members of the Chondrichthyes class (cartilaginous fishes).

The antibody (e.g. IgG, IgA, IgM, IgE, or IgD) may be one or more of recombinant, fully human, humanized or humanized murine, chimeric.

The antibody may be whole antibody or a fragment. An antibody fragment may comprise an antigen binding domain. Preferably, the antibody fragment displays antigen binding function. Antibody fragments include those known in the art, for example Fab, F(ab′)2, Fab3, scFv, Fv, dsFv, ds-scFv, Fd, dAbs, TandAbs, flexibodies dimers, minibodies, diabodies, tribodies, tetrabodies, vH domain, vL domain, vHH domain, nanobodies, IgNAR variable single domain (v-NAR domain), fragments thereof, and multimers thereof and bispecific antibody fragments.

Antibodies can be fragmented using conventional techniques. F(ab′)2 fragments can be generated by treating the antibody with pepsin and can be treated to reduce disulfide bridges to produce Fab fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can be synthesized by recombinant techniques or by chemical synthesis. Techniques for producing antibody fragments are well known and described in the art.

Preferably the antibody, antibody fragment, or antibody fusion comprises an antibody light chain variable region (vL) and/or an antibody heavy chain variable region (vH) which generally comprise the antigen binding site. The antibody, antibody fragment, or antibody fusion may comprise all or a portion of a heavy chain constant region, such as an IgG1, IgG2, IgG3, IgG4, IgA1, IgA2, IgE, IgM or IgD constant region. Preferably, the heavy chain constant region is an IgG1 heavy chain constant region. Furthermore, the antibody, antibody fragment or antibody fusion may comprise all or a portion of a kappa light chain constant region or a lambda light chain constant region. Preferably, the light chain constant region is a lambda light chain constant region. All or part of such constant regions may be produced naturally or may be wholly or partially synthetic. Appropriate sequences for such constant regions are known in the art.

The antibody, or antibody fragment, may be fused or conjugated to a ‘partner’ thus forming an antibody fusion or antibody conjugate. The partner may be a polypeptide having an amino acid sequence of at least about 10, 20, 30, 40, 50, 75 or at least about 100 residues. An antibody fusion may be a N-terminal fusion or a C-terminal fusion or a N- and C-terminal fusion understood as a fusion where the antibody sequence is fused N-, C- or N- and C-terminally to the partner sequence. The antibody sequence may also be inserted internally into the non-antibody sequence such as in a loop or a structure known to be located on the surface of the molecule comprising said non antibody sequence. The fusion may further comprise linker sequences between the antibody and partner sequences. This concept is outlined in WO 01/79442 (incorporated herein by reference in its entirety). Preferably, in an antibody fusion, the antibody component is a fragment, such as a Fab fragment, F(ab′)2 fragment or scFv fragment. The partner may be albumin, e.g. HSA, or a fragment thereof.

The antibodies, antibody fragments or antibody fusions may be produced recombinantly in a suitable host cell. The antibodies, antibody fragments or antibody fusions may be produced recombinantly in their final form or they may be produced in a form that can be converted into the final desired antibody, antibody fragment, or antibody fusion by one or more subsequent steps. For example, an antibody fragment according to the invention may be produced recombinantly as a whole antibody in a suitable host cell, and then converted into the desired antibody fragment using conventional techniques e.g. cleavage with a protease.

The antibody may be obtained from any suitable source. Preferably, the antibody is obtained from eukaryotic cell culture such as yeast or mammalian cell culture. Mammalian cell culture is preferred. Mammalian cells include COS cells, mouse L-cells, mouse C127-cells, hamster BHK-21 cells, human embryonic kidney 293 cells, hamster CHO cells, Vero or PERC6 cells. Preferred mammalian cells include Chinese Hamster Ovary (CHO), NSO murine myeloma cells, and PER.C6® human cells, particularly CHO cells.

The antibody may be monoclonal or polyclonal. Monoclonal antibodies are preferred because they may be highly specific, being directed against a single antigenic site. Monoclonal antibodies are typically obtained from a population of substantially homogeneous antibodies, i.e., the individual antibodies comprising the population are identical except for possible naturally occurring mutations that may be present in minor amounts. Monoclonal antibodies may be produced by the hybridoma method first described by Kohler et al., Nature 256:495 (1975), or by recombinant DNA methods (e.g. U.S. Pat. No. 4,816,567), or by phage antibody libraries (e.g. Clackson et al., Nature 352:624-628 (1991) and Marks metal, J. Mol. Biol. 222:581-597 (1991)).

The composition (e.g. liquid composition) may comprise 2 or more different antibodies, for example at least 3, 4, 5, 6, 7, 8, 9, 10, 11, 12, 13, 14 or 15 different antibodies. An advantage of a composition comprising several antibodies is that several antigens can be targeted. For example, a composition comprising several different antibodies may facilitate prevention or treatment of medical conditions by simultaneously targeting several different antigens or locations. This may improve efficacy and/or minimize development of drug resistance. Antibodies are useful for treating auto-immune conditions and cancers, and these are often caused by defects in the immune system. Therefore, targeting multiple targets reduces the likelihood of the patient's immune system evading the treatment by use of an alternative part of the immune system. The 2 or more different antigens may be from the same class (e.g. IgG, IgA, IgM, IgE or IgD) and/or subclass (e.g. IgG1, IgG2, IgG3 or IgG4) or from 2, 3 or 4 different classes and/or subclasses. A composition in which all antigens are in the IgG class, particularly IgG1, is preferred.

The antibody may comprise 2 light chains each of from about 200 to about 230 amino acids, such as from about 210 to about 220 amino acids, particularly about 214 or about 213 amino acids.

The antibody may comprise 2 heavy chains each of from about 425 to about 475 amino acids, such as from about 450 to about 455 amino acids, particularly about 451 or about 453 amino acids.

The antibody may bind to VEGF and/or inhibit binding of VEGF to Flt-1 (VEGFR-1) and/or inhibits binding of VEGF to KDR (VEGFR-1).

The antibody may bind to the transmembrane antigen CD20 for example on pre-B and mature-B lymphocytes. The antibody may not bind to CD20 on haemopoietic stem cells, pro-B-cells, normal plasma cells or other normal tissue. Following antibody binding, CD20 is not internalized or shed from the cell membrane into the environment. CD20 does not circulate in the plasma as a free antigen and, thus, does not compete for antibody binding

Suitable antibodies include 3F8, 8H9, B7-H3, Abagovomab, Abciximab, Actoxumab, Adalimumab, Adecatumumab, Afelimomab, Afutuzumab, Alacizumab pegol, Alemtuzumab, Alirocumab, Amatuximab, Anatumomab mafenatox, Anifrolumab, Anrukinzumab, Apolizumab, Arcitumomab, Aselizumab, Atinumab, Atlizumab (Tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Zenapax, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Raptiva, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Inolimomab, lnotuzumab ozogamicin, Intetumumab, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Matuzumab, Mavrilimumab, Mepolizumab, Metelimumab, Milatuzumab, Minretumomab, Mitumomab, Mogamulizumab, Morolimumab, Motavizumab, Moxetumomab pasudotox, Muromonab-DC3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, Ticilimumab (Tremelimumab), Tigatuzumab, Tildrakizumab, Tocilizumab, Toralizumab, Tositumomab (e.g. Iodine 131 Tositumomab), Tovetumab, Tralokinumab, Trastuzumab, Ektomab, Tregalizumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, and Zolimomab aritox.

Preferred antibodies include Abciximab, Adalimumab, Alemtuzumab, Basiliximab, Bevacizumab, Canakinumab, Certolizumab pegol, Cetuximab, Daclizumab, Denosumab, Eculizumab, Golimumab, Ibritumomab tiuxetan, Infliximab, Muromonab-DC3, Natalizumab, Ofatumumab, Omalizumab, Palivizumab, Panitumumab, Ranibizumab, Rituximab, Tocilizumab, Tositumomab and Iodine 131 Tositumomab, Trastuzumab, and Ustekinumab, particularly Bevacizuab and Rituximab.

The albumin concentration of the composition (e.g. liquid composition) may be at least about 10 mg/mL, such as at least about 10, 20, 30, 40, or 50 mg/mL. The albumin concentration may be from about 10, 20, 30, 40, 50, 60, 70, 80, 90 to about 20, 30, 40, 50, 60, 70, 80, 90 or 100 mg/mL. The albumin concentration may be from about 10 to about 75 mg/mL, such as from about 10 to about 20 mg/mL for example from about 10, 12, 14, 16, or 18 to 12, 14, 16, 18 or 20 mg/mL. The albumin concentration may be from about 30 to about 70 or from about 40 to about 60 mg/mL.

The ratio (e.g. weight or molar ratio) of albumin to antibody (albumin: antibody) may be at least 1:50, at least 1:10, at least 1:5, at least 1:4, at least 1:3, at least 1:2, at least 3:2, at least 2:3, or at least 1:1.

The antibody may have a formulation comprising about the following relative amounts: 100 mg antibody, 240 mg α,α-trehalose dihydrate, 23.2 mg sodium phosphate (monobasic, monohydrate), 4.8 mg sodium phosphate (dibasic, anhydrous), 1.6 mg polysorbate 20, and water for injection (USP) to make a volume of 4 mL.

The antibody may have a formulation comprising the following relative amounts: 400 mg antibody, 960 mg α,α-trehalose dihydrate, 92.8 mg sodium phosphate (monobasic, monohydrate), 19.2 mg sodium phosphate (dibasic, anhydrous), 6.4 mg polysorbate 20, and water for injection (USP) to make a final volume of 4 mL. The antibody formulation may be about pH 6.2.

The antibody may have a formulation comprising about the following relative amounts: 10 mg/mL antibody, 0.7 mg/mL polysorbate 80, 7.35 mg/mL sodium citrate dehydrate, 9 mg/mL sodium chloride and water for injection to a desired final volume such as 10 mL or 50 mL. The antibody formulation may be about pH 6.5.

It is preferred that the antibody formulation comprises a salt and/or albumin.

The albumin may have at least 80% sequence identity to SEQ ID NO: 2, such as at least 85, 90, 95, 96, 97, 98, 99, 99.2, 99.4, 99.6, 99.8, 99.9% identity to SEQ ID NO: 2. The albumin may differ from SEQ ID NO: 2 by up to 10 amino acids, e.g. up to 1, 2, 3, 4, 5, 6, 7, 8, 9 or 10 amino acids. The term ‘differ’ includes deletion or substitution of amino acids. Substitution may be conservative or non-conservative. The albumin may have 100% identity to SEQ ID NO: 2. The albumin may consist of SEQ ID NO: 2.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

The albumin may be obtained from any suitable source including a mammalian source such as blood, serum or plasma, particularly human blood, serum or plasma; a recombinant source such as a recombinant eukaryote or prokaryote. Albumin obtained from a recombinant host is referred to as recombinant albumin (‘rAlbumin’).

Serum derived HSA has previously been used to stabilize peptides both in solution and in lyophilized state. As the most abundant protein in human plasma, the potential for HSA to illicit an immunogenic response is minimal, making it an ideal excipient candidate. However, serum derived HSA has the disadvantage of being derived from donated human blood with the attendant risk of contamination with infection agents. Hence recombinant HSA is preferred in the present invention.

Albumin suitable for use in the present invention may be encoded by a nucleotide sequence, for example SEQ ID NO: 1.

An antibody suitable for use in the present invention may be encoded by a nucleotide sequence which can be determined by the skilled person with reference to amino acid sequences SEQ ID NO: 3 and 4 (Bevacuzimab: light and heavy chain, respectively) or SEQ ID NO: 5 and 6 (Ritixumab: light and heavy chain, respectively). SEQ ID NO: 4 optionally comprises N-linked oligosaccharide. The antibody may comprise one or more chains have at least 80% sequence identity to SEQ ID NO: 3, 4, 5 or 6, such as at least 85, 90, 95, 96, 97, 98, 99, 99.2, 99.4, 99.6, 99.8, 99.9% identity to SEQ ID NO: 3, 4, 5 or 6. The antibody may differ from SEQ ID NO: 3, 4, 5 or 6 by up to 10 amino acids, e.g. up to 1, 2, 3, 4, 5, 6, 7, 8, 9 or 10 amino acids. The term ‘differ’ includes deletion or substitution of amino acids. Substitution may be conservative or non-conservative. The antibody may have 100% identity to SEQ ID NO: 3, 4, 5, or 6 such as an antibody comprising or consisting of 2 light chains according to SEQ ID NO: 3 and 2 heavy chains according to SED ID NO: 4, or comprising or consisting of 2 light chains according to SEQ ID NO: 5 and 2 heavy chains according to SED ID NO: 6. The antibody may consist of SEQ ID NO: 3, 4, 5 or 6.

Techniques for expressing a polynucleotide in a host cell are known in the art. Modification of a polynucleotide encoding a polypeptide of the present invention may be necessary for synthesizing polypeptides substantially similar to the polypeptide. The term ‘substantially similar’ to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, e.g., a subsequence thereof, and/or by introduction of nucleotide substitutions that do not result in a change in the amino acid sequence of the polypeptide, but which correspond to the codon usage of the host organism intended for production of the albumin, or by introduction of nucleotide substitutions that may give rise to a different amino acid sequence. For a general description of nucleotide substitution, see, e.g., Ford et al., 1991, Protein Expression and Purification 2: 95-107.

The polynucleotide may be expressed in a nucleic acid construct comprising the polynucleotide operably linked to one or more control sequences that direct the expression of the coding sequence in a suitable host cell under conditions compatible with the control sequences. The polynucleotide or nucleic acid construct may be within an expression vector.

The polynucleotide, nucleic acid construct or vector may be within a host cell such as a recombinant host cell. The host cell may be any cell useful in the recombinant production of the albumin or antibody, e.g., a prokaryote or a eukaryote.

The albumin or antibody may be obtained from a recombinant host cell such as eukaryotic cell. Suitable eukaryotic cells include mammalian cells, fungal cells and plant cells. Mammalian cells include monkey COS cells, mouse L-cells, mouse C127-cells, hamster BHK-21 cells, human embryonic kidney 293 cells, hamster CHO cells, Vero or PERC6 cells. For antibodies, CHO cells are particularly preferred.

Albumins have been successfully expressed as recombinant proteins in a range of hosts including fungi (including but not limited to Aspergillus (WO06066595), Kluyveromyces (Fleer 1991, Bio/technology 9, 968-975), Pichia (Kobayashi 1998 Therapeutic Apheresis 2, 257-262) and Saccharomyces (Sleep 1990, Bio/technology 8, 42-46)), bacteria (Pandjaitab 2000, J. Allergy Clin. Immunol. 105, 279-285)), animals (Barash 1993, Transgenic Research 2, 266-276) and plants (including but not limited to potato, rice (e.g. Oryza sativa) and tobacco (Sijmons 1990, Bio/technology 8, 217 and Farran 2002, Transgenic Research 11, 337-346) and mammalian cell lines such as CHO cells (WO 2007/090584). Therefore the albumin used in the present invention may be obtained from such as source.

The albumin may comprise from about 100 to about 1000 mM metal cations such as sodium. For example, the albumin may comprise from about 120 to about 160 mM metal cations, e.g. about 145 mM cations.

The albumin may comprise from about 0 to about 50 mM octanoate such as about 2 to about 40 mM octanoate e.g. about 4 to about 12 mM octanoate or about 28 to about 36 mM octanoate, preferably about 8 mM or about 32 mM octanoate.

The albumin may comprise from about 0 to about 75 mg/L of a surfactant such as a detergent (e.g. polysorbate 80 or polysorbate 20 mg/L) such as about 10 to about 20 mg/L. About 15 mg/L surfactant or about 50 mg/L surfactant is preferred.

The albumin or antibody composition may comprise less than or equal to about 0.5 EU/mL endotoxin, e.g. as measured by Limulus amebocyte lysate (LAL) testing. The albumin and/or the antibody composition may have a pH from about 5 to about 8.5, such as from about 5.5 to about 7.5, preferably from about 6 to about 7. The albumin may have a purity of at least 95, more preferably at least 99% as measured by native polyacrylamide gel electrophoresis (PAGE). The albumin may comprise less than or equal to 5, 4, 3, 2, or less than 1% polymer e.g. as measured by gas-phase high-performance liquid chromatography (GP HPLC). The albumin may comprise less than about 200 ng host cell protein per gram of albumin as measured by ELISA e.g. YA53M less than or equal to about 15 ng/g albumin and/or YA53H less than or equal to about 150 ng/g albumin. The albumin may comprise less than or equal to about 0.30% (w/w/) ConA-binding-albumin. The albumin may comprise less than or equal to about 0.5 μg (microgram) nickel per gram albumin. The albumin may comprise less than or equal to about 0.01 nmol potassium per gram albumin.

A preferred albumin includes recombinant albumin obtained from Saccharomyces cerevisiae having a sodium content of about 130 to about 160 mM (e.g. about 145 mM), an octanoate content of about 28.8 to about 35.2 mM (e.g. about 32 mM) and pH from about 6.7 to about 7.3. Such a preferred albumin includes Recombumin® Prime (Novozymes Biopharma), for example provided at about 20 mg/mL.

Another preferred albumin includes recombinant albumin obtained from Saccharomyces cerevisiae having the characteristics described in WO2013/006675 (incorporated herein by reference in its entirety) for example having a sodium content of from about 225 to about 275 mM (e.g. about 250 mM), a phosphate concentration of from about 20 to about 30 mM (e.g. about 25 mM), a pH from about 6.0 to about 7.0 (e.g. about 6.5) and an octanoate concentration of less than about 2 mM. Such an albumin may be provided at about 10 mg/mL. WO2013/006675 is incorporated herein by reference in its entirety.

Yet another preferred albumin includes recombinant albumin obtained from Saccharomyces cerevisiae having a sodium content of about 120 to about 160 mM (e.g. about 145 mM), an octanoate content of about 4 to about 12 mM (e.g. about 8 mM) and pH from about 6.4 to about 7.4. Such a preferred albumin includes Recombumin® Alpha (Novozymes Biopharma), for example provided at about 10 mg/mL.

The composition (e.g. liquid composition) may comprise one or more (several) excipient, for example a non-reducing sugar such as sucrose or mannitol or, more preferably, trehalose (e.g. trehalose dehydrate), metal salt (e.g. sodium phosphate, sodium citrate, sodium chloride), surfactant such as a detergent (e.g. polysorbate 20 or polysorbate 80), inorganic acid (e.g. hydrochloric acid), inorganic base (e.g. sodium hydroxide), water for injection.

In the composition (e.g. liquid composition), salt (e.g. NaCl) may be present at a concentration from 0 to 20% such as from about 0, 1, 2, 3, 4, 5, 10, 15 to about 1, 2, 3, 4, 5, 10, 15, 20%. Alternatively, the level of salt in the composition may be defined by molarity. A molarity of from about 0 to about 200 mM, for example from about 0, 5, 10, 15, 20, 25, 50, 75, 100, 125, 150, or 175 to about 5, 10, 15, 20, 25, 50, 75, 100, 125, 150, 175 or 200 mM is preferred. A molarity of at least 10 to 15 mM is preferred. A molarity less than 185, more preferably less than 150 mM is preferred. An isotonic level of salt is preferred. Inclusion of a salt, e.g. NaCl, may reduce viscosity of the liquid formulation.

Non-reducing sugar (e.g. sucrose or, more preferably, trehalose) or a sugar alcohol (e.g. mannitol) may be present at a concentration from 0 to 10%, such as from about 0, 1, 2, 3, 4, 5, 6, 7, 8, or 9 to about 2, 3, 4, 5, 6, 7, 8 or 10%. An isotonic level of non-reducing sugar is preferred, for example about 4 to about 6%, preferably about 5%, trehalose. Trehalose may be obtained from any suitable source including Saccharomyces cerevisiae.

The composition (e.g. liquid composition) may comprise surfactant, such as detergent (e.g. polysorbate 20 or polysorbate 80). Surfactant may be present at a concentration of from about 0 to about 1% (v/v), preferably from about 0 to about 0.1% such as from about 0 to about 0.01 or from about 0 to about 0.001% (v/v).

The composition (e.g. liquid composition) may have a pH in the range from about 5.0 to about 8.5, preferably from about 5.5. 7.5, most preferably from about 6.0 to about 7.0.

The composition (e.g. liquid composition) may comprise a buffer. The buffer component may be present a concentration from about 0 to about 50 mM, such as from about 0, 5, 10, 15, 20, 25, 30, 35, 40, or 45 mM to about 5, 10, 15, 20, 25, 30, 35, 40, 45 or 50 mM. Suitable buffers include histidine, phosphate, citrate and acetate. Histidine buffers are preferred because of their ability to stabilize proteins at around neutral pH. Phosphate buffers are less preferred. Preferably, the buffer has a pH in the range from about 5.0 to about 8.5, preferably from about 5.5. 7.5, most preferably from about 6.0 to about 7.0.

The composition (e.g. liquid composition) may comprise one or more (several) preservatives. Alternatively, the composition (e.g. liquid composition) may be essentially free of preservative. Inclusion of a preservative is useful, for example, for multi-dose preparations of the composition (e.g. liquid composition) of antibody. Preservative may be included at a level from about 0.1 to about 2% (v/v) for example from about 0.5 to about 1% (v/v).

The composition (e.g. liquid composition) may comprise octanoate for example from about 0 to about 20 mM octanoate, such as from about 0, 1, 2, 3, 4, 5, 6, 7, 8, 9, 10, 12, 14, 16, or 18 to about 2, 3, 4, 5, 6, 7, 8, 9, 10, 12, 14, 16, 18 or 20 mM octanoate. An octanoate content of from about 2 to 16, such as about 2 to 8, especially about 2 to 3 mM is preferred.

The antibody content of the composition (e.g. liquid composition) may comprise at least 90% monomer, preferably at least 91, 92, 93, 94, 95, 96, 97, 98, 99, 99.1, 99.2, 99.3, 99.4, 99.5, 99.6, 99.7, 99.8 or at least 99.9% monomer. The antibody content of the composition (e.g. liquid composition) may comprise at most 10% antibody aggregates (such as dimers, multimers, polymers and/or particulates of antibodies), preferably at most 9, 8, 7, 6, 5, 4, 3, 2, 1, 0.9, 0.8, 0.7, 0.6, 0.5, 0.4, 0.3, 0.2 or at most 0.1% antibody aggregates. Preferably monomer, dimer and polymer content is measured by AF4, such as described in Example 1. Preferably particulate content is measured by MFI, such as described in Example 6.

The composition (e.g. liquid composition) may be sterile. Sterility may be achieved by filtration e.g. with a 2 micron filter.

A second aspect of the invention provides a method for producing a composition (e.g. liquid composition) of antibody comprising combining antibodies with albumin to produce a composition having an antibody concentration greater than or equal to 140 mg/mL and an albumin concentration greater than or equal to about 10 mg/mL. Options and preferences for the first aspect of the invention apply to the second aspect of the invention.

Prior to the combining of the antibodies and the albumin, the antibodies may be in a liquid state or in a lyophilized state. Antibodies may be obtained from a source at a relatively low concentration and it may be desirable to increase the concentration in order to produce a composition (e.g. liquid composition) according to the present invention. Therefore, prior to combining, there may be a step of increasing the concentration of an antibody. Alternatively, the antibody may be provided at a suitable concentration. Suitable concentrations include at least 140 mg/mL, e.g. from about 140, 150, 160, 170, 180, 190, 200, 225, 250, 275, 300, 350, 400, or about 450 to about 150, 160, 170, 180, 190, 200, 225, 250, 275, 300, 350, 400, 450, or about 500 mg/mL. Preferred concentrations include from about 140 to about 350 mg/mL, from about 140 to 250 mg/mL and from about 140 to 200 mg/mL.

Lyophilization is useful to increase the concentration of antibody for use in this method. Other suitable methods for increasing the concentration include osmotic driven (dialysis) methods such as diafiltration against solutions or against water absorbing materials; solvent evaporation, such as vacuum, nitrogen flow or lyophilization; precipitation such as using solvent or salt or super-critical fluid (SCF) processes; freezing; chromatographic binding and elution and/or filtration such as centrifugal filtration, pressure filtration or tangential flow filtration (Shire et al (2004), op. cit.). Lyophilization is preferred.

The composition may or may not be subjected to a method or methods to increase the concentration of antibody and, if present, other constituents.

The composition may or may not be subjected to lyophilization and then reconstituted e.g. resuspended in liquid to provide a composition of a final desired concentration, such lyophilization can be beneficial to providing compositions of high concentrations. For example, a composition of antibody, albumin, liquid (e.g. water or buffer) and optionally other constituents may be prepared (step 1), the composition lyophilized (step 2) and then the lyophilized composition reconstituted in a liquid (e.g. water or buffer) (step 3). The liquid used for resconstituting (step 3) may be the same or different to the liquid used for the initial preparation of the composition (step 1). A lyophilization step may or may not include the presence of a non-reducing sugar such as trehalose or sucrose as a lyoprotectant.

The antibody may be obtained from a pharmaceutical preparation, such as a pharmaceutical preparation containing from 5 to 100 mg/mL.

Combining includes ‘mixing’. It is preferred that mixing is gentle to control or reduce the risk of antibody aggregation.

The method may include lyophilizing the composition. Lyophilization techniques are known in the art (Shire, 2004, op. cit.)

The method may include filling the composition into a container such as a syringe, vial or bottle.

The method may comprise reconstituting the lyophilized antibody-albumin composition in a suitable liquid such as a buffer, following lyophilization or following filling into a container.

The method may include sterilizing the composition, for example by filtering e.g. with a 0.2 micron filter. The sterilizing step may be carried out before or after filling into the container.

Alternatively, the method may comprise combining a purified lyophilized antibody with albumin and optionally filling the resultant mixture into a container.

The container may be comprised of any suitable material, for example glass or polymer such as a plastic. Suitable plastic types include cyclo olefin polymer (COP) and cyclo olefin co polymer (COC). COCs are clear amorphous copolymers based on cyclic and linear olefins, they may have high transparency, low density, good moisture barrier capabilities, and resistance to aqueous and polar organic media. Suitable COCs include those available from Topas Advanced Polymers (Frankfurt-Hoechst, Germany).

The invention also provides a method for inhibiting and/or preventing aggregation of an antibody in a composition (e.g. liquid composition). For example, the method for inhibiting and/or preventing may be according to the second aspect of the invention. Inhibition may be partial, e.g. resulting in at least about 1, 2, 3, 4, 5, 10, 15, 20, 25, 30, 35, 40, 45, 50, 55, 60, 65, 70, 75, 80, 85, 90, 95 or 99% fewer aggregates than a reference composition which is identical to the test composition except that it does not contain albumin. Inhibition may be partial, e.g. resulting in at most about 99, 98, 97, 95, 95, 90, 85, 80, 75, 70, 65, 60, 55, 50, 45, 40, 35, 30, 25, 20, 15, 10, 9, 8, 7, 6, 5, 4, 3, 2, 1% of the number of aggregates contained in reference composition which is identical to the test composition except that it does not contain albumin.

A third aspect of the invention provides use of albumin to stabilize an antibody composition in which antibody is present at a concentration of greater than or equal to about 140 mg/mL. Options and preferences for the first and second aspects of the invention apply to the second aspect of the invention.

A fourth aspect of the invention provides an antibody preparation, according to the first aspect of the invention or obtained by the second or third aspect of the invention, for treatment or prevention of disease or of a medical condition. The composition (e.g. liquid composition) may be suitable for administration, or administered, to a patient by any suitable route. Suitable routes include subcutaneous, intravenous administration (e.g. as a bolus or by continuous infusion over a period of time), intramuscular, intraperitoneal, intracerobrospinal, intra-articular, intrasynovial, intrathecal, oral, topical, or inhalation. Subcutaneous or intravenous administration is preferred. Subcutaneous administration is most preferred. The composition (e.g. liquid composition) may be administered by syringe or by intravenous line.

The antibody may be Bevacizumab and the disease or medical condition may be selected from the group consisting of: metastatic colorectal cancer; advanced and/or metastatic renal cell cancer; advanced, metastatic or recurrent non-squamous Non-Small Cell Lung Cancer; metastatic breast cancer; relapsed high grade malignant glioma; or epithelial ovarian, fallopian tube or primary peritoneal cancer. The Bevacizumab antibody preparation may comprise one or more other pharmacologically active compounds such as:
a) a chemotherapy agent such as:
   i) a fluoropyrimidine medicine (e.g. for treatment of advanced cancer in the large bowel e.g. in the colon or rectum);
   ii) paclitaxel or capecitabine (e.g. for treatment of breast cancer such as metastatic breast cancer);
   iii) a chemotherapy regimen containing platinum (e.g. for treatment of advanced non-small cell lung cancer).
b) interferon (e.g. for treatment of kidney cancer such as advanced kidney cancer).
c) carboplatin and/or paclitaxel (e.g. for treatment of advanced epithelial ovarian, fallopian tube, or primary peritoneal cancer).
d) carboplatin and gemcitabine (e.g. for treatment of those patients whose disease has come back at least 6 months after the last time they were treated with a chemotherapy regimen containing a platinum).

The antibody preparation may be Rituximab and the disease or medical condition may be selected from the group consisting of: Non-Hodgkin's lymphoma (NHL), Chronic lymphocytic leukemia (CLL), Rheumatoid arthritis, Granulomatosis with polyangiitis and Microscopic polyangiitis. The Rituximab antibody preparation may further comprise one or more other pharmacologically active compounds such as:
a) chemotherapy agent (e.g. for treatment of Non-Hodgkin's Lymphoma e.g. stage III-IV follicular lymphoma)
b) combination of chemotherapy agents e.g. cyclophosphamide, doxorubicin, vincristine, and prednisolone (e.g. for treatment of patients with CD20 positive diffuse large B cell non-Hodgkin's lymphoma)
c) glucocorticoids (e.g. for induction of remission in adult patients with severe, active Granulomatosis with polyangiitis (Wegener's) (GPA) and Microscopic polyangiitis (MPA)).

A fifth aspect of the invention provides a container holding an antibody preparation according to the first or fourth aspect of the invention or obtained by the second or third aspect of the invention. The container may be a syringe, vial or bottle, for example a pre-filled syringe. The container may be a single-use or a multi-use container. The container may be or comprise an auto injector such as a spring-loaded syringe. The auto injector may be single use or multi-use.

The container may contain a therapeutically effective amount of antibody to provide a single dose of antibody to a patient such as a 10 to 150 kg patient, e.g. a patient from about 10, 20, 30, 40, 50, 60, 70, 80, 90, 100, 110, 120, 130, 140 to about 20, 30, 40, 50, 60, 70, 80, 90, 100, 110, 120, 130, 140 kg, preferably from about 50 to about 100 kg.

The container may contain a therapeutically effective amount of antibody to provide multiple doses (e.g. at least 2, 3, 4 or 5 doses) of antibody to a patient such as a 10 to 150 kg patient, e.g. a patient from 10, 20, 30, 40, 50, 60, 70, 80, 90, 100, 110, 120, 130, 140 to about 20, 30, 40, 50, 60, 70, 80, 90, 100, 110, 120, 130, 140 kg, preferably from about 50 to about 100 kg.

There may be about 0.5 to about 20 mL antibody composition (e.g. liquid antibody composition) in the container, e.g. from about 0.5, 1, 2, 3, 4, 5, 6, 7, 8, 9, 10, or 15 to about 1, 2, 3, 4, 5, 6, 7, 8, 9, 10, 15 or 20 mL.

The syringe volume may be about 1 mL to about 10 mL such as from about 1, 2, 3, 4, or 5 mL to about 2, 3, 4, 5 or 10 mL. The syringe may be used with a needle such as with a gauge of from 15 to 34 or higher (i.e. narrower diameter), preferably from 20 to 34 or higher. A needle gauge of about 25 to about 35, such as about 27 is preferred.

A sixth aspect of the invention provides a kit comprising a container according to the fifth aspect of the invention and instructions such as administration and/or dosage instructions, optionally in the form or a leaflet or electronic storage device.

A seventh aspect of the invention provides for use of albumin to decrease the injection force of an antibody composition e.g. relative to the injection force expected, based on protein concentration, for a composition comprising antibody and albumin. Decreased injection force is advantageous to increase the ease of administration of the composition to a patient and/or to decrease the pain associated with administration to a patient, while the presence of albumin is advantageous because it increases the stability of the antibody.

The composition may comprise antibody at a concentration of greater than or equal to about 140 mg/mL and albumin at a concentration of greater than or equal to about 1 mg/mL.

The composition may have an injection force no more than 100% of the injection force of an equivalent composition in which the albumin component is replaced with antibody. The injection force may be less than or equal to 90, 80, 70, 60, 50, 50, 45, 40, 35, 30, or 25% of the injection force of an equivalent composition in which the albumin component is replaced with antibody. The injection force may be no more than 100% of the injection force predicted for an equivalent composition in which the albumin component is replaced with antibody. The injection force may be less than or equal to 90, 80, 70, 60, 50, 50, 45, 40, 35, 30, or 25% of the predicted injection force. The injection force may be below 25 N, preferably below 20, 19, 18, 17, 16, 15, 14, 13, 12, 11, 10, 9, 8, 7, 6, 5, 4, 3, 2, or 1 N. Injection force may be measured by extruding the composition through a 27 G ½″ needle at 2 or 4 mL/minute.

An eighth aspect of the invention provides for use of albumin to decrease the viscosity of an antibody composition e.g. relative to the viscosity expected, based on protein concentration, for a composition comprising antibody and albumin. Decreased viscosity is advantageous to improve manufacturing of an antibody composition, while the presence of albumin is advantageous because it increases the stability of the antibody.

The composition may have a viscosity no more than 100% of the viscosity of an equivalent composition in which the albumin component is replaced with antibody. The viscosity may be less than or equal to 90, 80, 70, 60, 50, 50, 45, 40, 35, 30, or 25% of viscosity of an equivalent composition which does not include albumin. The viscosity may be no more than 100% of the viscosity predicted for an equivalent composition in which the albumin component is replaced with antibody. The viscosity may be less than or equal to 90, 80, 70, 60, 50, 50, 45, 40, 35, 30, or 25% of the predicted viscosity. The viscosity may be below 500 mPa·s, preferably below 450, 400, 350, 300, 250, 200, 150, 100, 50, 40, 30, or 20 mPa·s. The viscosity may be measured by rheometry (e.g. Anton Paar rheometer MCR 301) at 25° C., shear rate 10-10000 1/s using CP25-1 geometry.

The invention is further defined in the following embodiments:

### Embodiments

1) A composition (e.g. liquid composition) of antibody, the composition comprising antibody at a concentration of greater than or equal to about 140 mg/mL and albumin at a concentration of greater than or equal to about 10 mg/mL.
2) The composition (e.g. liquid composition) according to embodiment 1 in which the antibody concentration is from about 140 to about 300 mg/mL.
3) The composition (e.g. liquid composition) according to embodiment 1 or 2 in which the antibody concentration is from about 140 to about 250 mg/mL.
4) The composition (e.g. liquid composition) according to any preceding embodiment in which the antibody concentration is from about 140 to about 200 mg/mL.
5) The composition (e.g. liquid composition) according to any preceding embodiment in which the antibody concentration is from about 140 to about 175 mg/mL.
6) The composition (e.g. liquid composition) according to any preceding embodiment in which, following exposure to stress for about one month, the composition (e.g. liquid composition) contains at least 1 percentage point fewer antibody aggregates, e.g. dimers, multimers, polymers and/or particulates, compared with a reference composition which is identical to the antibody composition with the exception that it does not comprise albumin, or does not comprise albumin at a concentration of at least about 10 mg/mL.
7) The composition (e.g. liquid composition) according to embodiment 6 in which, following exposure to stress for about one month the composition (e.g. liquid composition) contains at least 2, 3, 4, 5, 6, 7, 8, 9, 10, 15, 20, 25, 30, 35, 40, 45, 50, 55, 60, 65, 70, 75, 80, 85, 90, 95, 96, 97, 98 or 99 percentage points fewer antibody aggregates such as dimers, multimers, polymers and/or particulates (e.g. particulates of at least 1 micrometer size such as from 1 to 100, 2 to 25 or preferably 2 to 10 micrometer size) compared with a reference composition which is identical to the antibody composition with the exception that it does not comprise albumin or contains at most about 99, 98, 97, 96, 95, 90, 85, 80, 75, 70, 65, 60, 55, 50, 45, 40, 35, 30, 25, 20, 15, 10, 5, 4, 3, 2, 1, 0.1, 0.01% of the aggregates formed in the reference composition.
8) The composition (e.g. liquid composition) according to embodiment 6 or 7 in which the stress is selected from the group consisting of elevated temperature (e.g. about 25° C. or about 40° C.); shear; freeze thaw; and exposure to hydrophobic surfaces, preferably exposure to 40° C. for at least 28 days, at least 29 days or at least 63 days.
9) The composition according to embodiment 8 in which freeze thaw comprises freezing to about −20° C. or about −70° C. for 1 or more hours, followed by thawing at about 20° C. for 1 hour.
10) The composition (e.g. liquid composition) according to embodiment 8 or 9 in which the aggregates, such as antibody dimers, multimers, polymers and/or particulates (e.g. visible and/or sub-visible particles), are detected and/or quantified by SE-HPLC, AF4, MFI or DLS, preferably dimers, multimers and polymers are measured by AF4, preferably particulates are measured by MFI.
11) The composition (e.g. liquid composition) according to embodiment 10 in which the particulates are from about 1, 2, 3, 4, 5, 10, 20, 25, 30, 40, 50 to about 2, 3, 4, 5, 10, 20, 25, 30, 40, 50, 60, 70, 80, 90, 100 μm (micrometers) diameter, preferably from 2 to 100, from 2 to 10, from 10 to 100 or from 25 to 100 μm (micrometers) diameter, most preferably 2 to 10 μm (micrometers) diameter.
12) The composition (e.g. liquid composition) according to embodiment 11 in which the particulates are visible.
13) The composition (e.g. liquid composition) according to embodiment 11 in which the particulates are sub-visible.
14) The composition (e.g. liquid composition) according to embodiment 11 in which the particulates are visible particulates and sub-visible particulates.
15) The composition (e.g. liquid composition) according to any preceding embodiment which is isotonic.
16) The composition (e.g. liquid composition) according to any preceding embodiment which is stable at a temperature of about 2 to about 8° C. (e.g. about 5° C.) for at least about one month.
17) The composition (e.g. liquid composition) according to embodiment 16 which is stable at a temperature of from about 2 to about 8° C. (e.g. about 5° C.) for at least about 2, 3, 4, 5 or 6 months.
18) The composition (e.g. liquid composition) according to embodiment 17 which is stable at a temperature of from about 2 to about 8° C. (e.g. about 5° C.) for at least about 1 year, 2 years, or 3 years.
19) The composition (e.g. liquid composition) according to any preceding embodiment which is stable following freezing and thawing.
20) The composition (e.g. liquid composition) according to any preceding embodiment which is stable at from about 25° C. or 30° C. for at least about one month.
21) The composition (e.g. liquid composition) according to embodiment 20 which is stable at about 25° C. or 30° C. for at least about 6 months.
22) The composition (e.g. liquid composition) according to embodiment 21 which is stable at about 25° C. or 30° C. for at least about 1 year.
23) A composition (e.g. liquid composition) of antibody, the composition comprising antibody at a concentration of greater than or equal to about 140 mg/mL and albumin at a concentration of greater than or equal to about 1 mg/mL.
24) The composition (e.g. liquid composition) according to any preceding embodiment which has an injection force no more than 100% of the injection force of an equivalent composition in which the albumin component is replaced with antibody.
25) The composition (e.g. liquid composition) according to any preceding embodiment in which the injection force is less than or equal to 90, 80, 70, 60, 50, 50, 45, 40, 35, 30, 25% of the injection force of an equivalent composition in which the albumin component is replaced with antibody.
26) The composition according to any preceding embodiment in which the injection force is no more than 100% of the injection force predicted by preparing antibody compositions of about 100, about 150 and about 200 mg·mL⁻¹, calculating a line, or curve, of best fit and predicting the injection force for a composition having a protein concentration between 100 and 200 mg·mL⁻¹ or extrapolating to predict the injection force for a composition having a protein concentration below 100 mg·mL⁻¹ or above 200 mg·mL⁻¹.
27) The composition (e.g. liquid composition) according to any preceding embodiment in which the injection force is less than or equal to 90, 80, 70, 60, 50, 50, 45, 40, 35, 30, 25% of the predicted injection force.
28) The composition (e.g. liquid composition) according to any preceding embodiment which has an injection force below 25 N, preferably below 20, 19, 18, 17, 16, 15, 14, 13, 12, 11, 10, 9, 8, 7, 6, 5, 4, 3, 2, 1 N.
29) The composition (e.g. liquid composition) according to any of embodiments 25 to 28 in which the injection force is measured by extruding the composition through a 27 G ½″ needle at 2 or 4 mL/minute
30) The composition (e.g. liquid composition) according to any preceding embodiment which has a viscosity no more than 100% of the viscosity of an equivalent composition in which the albumin component is replaced with antibody.
31) The composition (e.g. liquid composition) according to embodiment 30 in which the viscosity is less than or equal to 90, 80, 70, 60, 50, 50, 45, 40, 35, 30, 25% of viscosity of an equivalent composition which does not include albumin.
32) The composition according to embodiment 23, 30 or 31 in which the viscosity is no more than 100% of the viscosity predicted by preparing antibody compositions of about 100, about 150 and about 200 mg·mL⁻¹, calculating a line, or curve, of best fit and predicting the viscosity for a composition having a protein concentration between 100 and 200 mg·mL⁻¹ or extrapolating to predict the viscosity for a composition having a protein concentration below 100 mg·mL⁻¹ or above 200 mg·mL⁻¹.
33) The composition (e.g. liquid composition) according to embodiment 32 in which the viscosity is less than or equal to 90, 80, 70, 60, 50, 50, 45, 40, 35, 30, or 25% of the predicted viscosity.
34) The composition (e.g. liquid composition) according to any preceding embodiment which has a viscosity below 500 mPa·s, preferably below 450, 400, 350, 300, 250, 200, 150, 100, 50, 40, 30, or 20 mPa·s.
35) The composition (e.g. liquid composition) according to any of embodiments 30 to 34 in which the viscosity is measured by rheometry (e.g. Anton Paar rheometer MCR 301) at 25° C., shear rate 10-10000 1/s using CP25-1 geometry.
36) The composition (e.g. liquid composition) according to any preceding embodiment in which the antibody is monoclonal.
37) The composition (e.g. liquid composition) according to any of embodiments 1 to 36 in which the antibody is polyclonal.
38) The composition (e.g. liquid composition) according to any preceding embodiment in which the antibody is selected from the group consisting of IgG, IgA, IgM, IgE, and IgD.
39) The composition (e.g. liquid composition) according to embodiment 38 in which the antibody is an IgG.
40) The composition (e.g. liquid composition) according to embodiment 39 in which the IgG is selected from the group consisting of subclass 1, 2, 3, and 4.
41) The composition (e.g. liquid composition) according to embodiment 40 in which the IgG is in subclass 1.
42) The composition (e.g. liquid composition) according to any preceding embodiment in which the antibody has a molecular weight of from about 125 to about 175 kDa.
43) The composition (e.g. liquid composition) according to embodiment 42 in which the antibody has a molecular weight of from about 135 to about 165 kDa.
44) The composition (e.g. liquid composition) according to embodiment 43 in which the antibody has a molecular weight of from about 145 to about 155 kDa.
45) The composition (e.g. liquid composition) according to embodiment 44 in which the antibody has a molecular weight of about 150 kDa.
46) The composition (e.g. liquid composition) according to any preceding embodiment in which the antibody is selected from the group consisting of recombinant, fully human, humanized or humanized murine, chimeric antibodies and/or bispecific antibodies of fragments thereof.
47) The composition (e.g. liquid composition) according to any preceding embodiment in which the antibody is a whole antibody.
48) The composition (e.g. liquid composition) according to any preceding embodiment in which the antibody is an antibody fragment.
49) The composition (e.g. liquid composition) according to embodiment 48 in which the antibody fragment is a F(ab′)₂, Fab, Fab′ or scFv.
50) The composition (e.g. liquid composition) according to any preceding embodiment in which the antibody comprises 2 light chains each of from 200 to 230 amino acids, such as from 210 to 220 amino acids and 2 heavy chains each of from 425 to 475 amino acids, such as from 450 to 455 amino acids.
51) The composition (e.g. liquid composition) according to any preceding embodiment in which the composition comprises 2 or more different antibodies.
52) The composition (e.g. liquid composition) according to embodiment 51 in which the composition comprises 3, 4, 5, 6, 7, 8, 9, 10, 11, 12, 13, 14 or 15 different antibodies.
53) The composition (e.g. liquid composition) according to embodiment 51 or 52 in which the different antibodies are from the same class, where class is IgG, IgA, IgM, IgE or IgD.
54) The composition (e.g. liquid composition) according to embodiment 51 or 52 in which the different antibodies are from 2 different classes, where class is IgG, IgA, IgM, IgE or IgD.
55) The composition (e.g. liquid composition) according to embodiment 52 or 53 in which the different antibodies are from 3, 4 or 5 different classes, where class is IgG, IgA, IgM, IgE or IgD.
56) The composition (e.g. liquid composition) according to embodiment 53 in which the different antibodies are IgG and in the same subclass, where subclass is IgG1, IgG2, IgG3 or IgG4.
57) The composition (e.g. liquid composition) according to embodiment 56 in which the different antibodies are IgG and in subclass IgG1.
58) The composition (e.g. liquid composition) according to embodiment 57 in which the different antibodies are both, or all, IgG and both, or all, in subclass IgG2, IgG3, or IgG4.
59) The composition (e.g. liquid composition) according to embodiment 53 in which the different antibodies are IgG and in 2 or more different subclasses, where subclass is IgG1, IgG2, IgG3 or IgG4.
60) The composition (e.g. liquid composition) according to embodiment 53 in which the different antibodies are IgG and in 3 or 4 different subclasses, where subclass is IgG1, IgG2, IgG3 or IgG4.
61) The composition (e.g. liquid composition) according to any preceding embodiment in which the antibody binds to VEGF and/or inhibits binding of VEGF to Flt-1 (VEGFR-1) and/or inhibits binding of VEGF to KDR (VEGFR-1).
62) The composition (e.g. liquid composition) according to embodiment 61 in which the antibody (or one of the antibodies) comprises Bevacizumab.
63) The composition (e.g. liquid composition) according to any of embodiments 1 to 60 in which the antibody binds to the transmembrane antigen CD20 for example on pre-B and mature-B lymphocytes.
64) The composition (e.g. liquid composition) according to embodiment 62 in which the antibody does not bind to CD20 on haemopoietic stem cells, pro-B-cells, normal plasma cells or other normal tissue.
65) The composition (e.g. liquid composition) according to embodiment 63 or 64 in which the antibody (or one of the antibodies) comprises Rituximab.
66) The liquid composition according to any preceding embodiment in which the antibody is obtained from mammalian cell culture.
67) The composition (e.g. liquid composition) according to embodiment 66 in which the mammalian cell is selected from the group consisting of Chinese Hamster Ovary (CHO), NSO murine myeloma cells, and PER.C6® human cells.
68) The composition (e.g. liquid composition) according to any preceding embodiment in which the albumin concentration is from about 10 to about 100 mg/mL.
69) The composition (e.g. liquid composition) according to embodiment 68 in which the albumin concentration is from about 25 to about 75 mg/mL.
70) The composition (e.g. liquid composition) according to embodiment 69 in which the albumin concentration is from about 40 to about 60 mg/mL.
71) The composition (e.g. liquid composition) according to embodiment 68 in which the albumin concentration is from about 10 to about 20 mg/mL.
72) The composition (e.g. liquid composition) according to any preceding embodiment in which the ratio (e.g. weight or molar ratio) of albumin to antibody (albumin:antibody) is at least 1:50, at least 1:10, at least 1:5, at least 1:4, at least 1:3, at least 1:2, at least 3:2, or at least 1:1.
73) The composition (e.g. liquid composition) according to any preceding embodiment in which the albumin has at least 80% sequence identity to SEQ ID NO: 2.
74) The composition (e.g. liquid composition) according to embodiment 73 in which the albumin has at least 90% sequence identity to SEQ ID NO: 2.
75) The composition (e.g. liquid composition) according to embodiment 74 in which the albumin has at least 95% sequence identity to SEQ ID NO: 2.
76) The composition (e.g. liquid composition) according to embodiment 75 in which the albumin has at least 99% sequence identity to SEQ ID NO: 2.
77) The composition (e.g. liquid composition) according to embodiment 76 in which the albumin has 100% sequence identity to SEQ ID NO: 2.
78) The composition (e.g. liquid composition) according to embodiment 77 in which the albumin sequence consists of SEQ ID NO: 2.
79) The composition (e.g. liquid composition) according to any preceding embodiment in which the albumin is obtained from a recombinant host.
80) The composition (e.g. liquid composition) according to embodiment 79 in which the recombinant host is a yeast such as Saccharomyces cerevisiae, Kluyveromyces lactis or Pichia pastoris.
81) The composition (e.g. liquid composition) according to embodiment 79 in which the recombinant host is a plant such as rice, e.g. Oryza sativa.
82) The composition (e.g. liquid composition) according to any of embodiments 1 to 78 in which the albumin is obtained from blood, serum or plasma of a mammal such as a human.
83) The composition (e.g. liquid composition) according to any preceding embodiment comprising from about 5 to about 200 mM sodium.
84) The composition (e.g. liquid composition) according to any preceding embodiment comprising from about 0 to about 20 mM octanoate.
85) The composition (e.g. liquid composition) according to any preceding embodiment in which surfactant, e.g. polysorbate 20 or polysorbate 80, is present at a concentration from about 0 to about 1%, preferably from about 0 to about 0.1 (w/v) %.
86) The composition (e.g. liquid composition) according to any preceding embodiment in which the albumin comprises less than or equal to about 0.5 EU/mL endotoxin, e.g. as measured by LAL.
87) The composition (e.g. liquid composition) according to any preceding embodiment in which the albumin and/or the resultant antibody composition has a pH from about 5 to about 8.5.
88) The composition (e.g. liquid composition) according to embodiment 87 in which the albumin and/or the resultant antibody composition has a pH from about 5.5 to about 7.5.
89) The composition (e.g. liquid composition) according to embodiment 88 in which the albumin and/or the resultant antibody composition has a pH from about 6 to about 7.
90) The composition (e.g. liquid composition) according to any preceding embodiment in which the albumin has a purity at least 99% as measured by native PAGE.
91) The composition (e.g. liquid composition) according to any preceding embodiment in which the albumin comprises less than or equal to about 1% polymer e.g. as measured by GP HPLC.
92) The composition (e.g. liquid composition) according to any preceding embodiment in which the albumin comprises less than about 200 ng host cell protein per gram of albumin as measured by ELISA e.g. YA53M less than or equal to about 15 ng/g albumin and/or YA53H less than or equal to about 150 ng/g albumin.
93) The composition (e.g. liquid composition) according to any preceding embodiment in which the albumin comprises less than or equal to about 0.30% (w/w/) ConA-binding-albumin.
94) The composition (e.g. liquid composition) according to any preceding embodiment in which the albumin comprises less than or equal to about 0.5 ug (microgram) nickel per gram albumin.
95) The composition (e.g. liquid composition) according to any preceding embodiment in which the albumin comprises less than or equal to about 0.01 nmol potassium per gram albumin.
96) The composition (e.g. liquid composition) according to any preceding embodiment further comprising an excipient selected from the group consisting of: non-reducing sugar, a sugar alcohol (e.g. mannitol), a metal salt (e.g. sodium phosphate, sodium citrate, sodium chloride), a surfactant such as detergent (e.g. polysorbate 20 or polysorbate 80), an inorganic acid (e.g. hydrochloric acid), an inorganic base (e.g. sodium hydroxide), water for injection.
97) The composition (e.g. liquid composition) according to embodiment 96 wherein salt (e.g. NaCl) is present at a concentration from about 0 to about 20%.
98) The composition (e.g. liquid composition or liquid formulation) according to any of embodiments 96 or 97 in which non-reducing sugar or sugar alcohol is present at a concentration from about 0 to about 25%, preferably about 0 to about 6%, more preferably about 4 to about 6% e.g. about 5%.
99) The composition (e.g. liquid composition or liquid formulation) according to embodiment 98 in which the non-reducing sugar is selected from the group consisting of trehalose and sucrose.
100) The composition (e.g. liquid composition) according to any preceding embodiment in which detergent (e.g. polysorbate 20 or polysorbate 80) is present at a concentration from about 0 to about 1%, preferably from about 0 to about 0.1% (w/v).
101) The composition (e.g. liquid composition) according to any preceding embodiment in which buffer component is present at a concentration from about 0 to about 50 mM.
102) The composition (e.g. liquid composition) according to embodiment 101 in which the buffer component is selected from the group consisting of histidine, phosphate, citrate and acetate.
103) The composition (e.g. liquid composition) according to any preceding embodiment in which the antibody content comprises at least 90% monomer, preferably at least 91, 92, 93, 94, 95, 96, 97, 98, 99, 99.1, 99.2, 99.3, 99.4, 99.5, 99.6, 99.7, 99.8 or at least 99.9% monomer.
104) The composition (e.g. liquid composition) according to any preceding embodiment in which the antibody content comprises at most 10% antibody aggregates (such as dimers, multimers, polymers and/or particulates of antibodies), preferably at most 9, 8, 7, 6, 5, 4, 3, 2, 1, 0.9, 0.8, 0.7, 0.6, 0.5, 0.4, 0.3, 0.2 or at most 0.1% antibody aggregates.
105) The composition (e.g. liquid composition) according to any preceding embodiment which has a pH from about 6 to 7, a cation concentration of from about 10 to about 185 mM, an octanoate concentration of from about 0 to about 16 mM and a detergent concentration of from about 0 to about 0.1 (w/v) %
106) A method for producing a composition of antibody comprising combining antibodies with albumin to produce a composition having an antibody concentration of greater than or equal to about 140 mg/mL and an albumin concentration is greater than or equal to about 10 mg/mL.
107) The method according to embodiment 106 in which the albumin, antibody or composition is according to any of embodiments 1 to 105.
108) The method according to embodiment 106 or 107 in which, prior to the combining step, the antibody is in a lyophilized state.
109) The method according to embodiment 106 or 107 in which, prior to the combining step, the antibody is in a liquid state.
110) The method according to any of embodiments 106 to 109, comprising lyophilizing the resultant composition.
111) The method according to claim 110, comprising reconstituting e.g. re-suspending the lyophilized composition.
112) The method according to any of embodiments 106 to 111, comprising sterilizing the composition.
113) The method according to any of embodiments 106 to 112, comprising filling the composition into a container.
114) Use of albumin to stabilize an antibody composition in which antibody is present at a concentration of greater than or equal to about 140 mg/mL.
115) The use according to embodiment 114 in which the albumin, antibody or composition is according to any of embodiments 1 to 105.
116) Use of albumin to control or reduce the injection force of a composition comprising antibodies.
117) Use according to embodiment 116 in which the resultant injection force of the composition is no more than 100% of the injection force of an equivalent composition in which the albumin component is replaced with antibody.
118) Use according to embodiment 116 or 117 in which the antibody is at a concentration of greater than or equal to about 140 mg/mL and the albumin is at a concentration of greater than or equal to about 1 mg/mL.
119) Use according to any of embodiments 116 to 118 in which the injection force of the composition is less than or equal to 90, 80, 70, 60, 50, 50, 45, 40, 35, 30, 25% of the injection force of an equivalent composition in which the albumin component is replaced with antibody.
120) Use according to any of embodiments 116 to 119 in which the injection force of the composition is no more than 100% of the injection force predicted by preparing antibody compositions of about 100, about 150 and about 200 mg·mL⁻¹, calculating a line, or curve, of best fit and predicting the injection force for a composition having a protein concentration between 100 and 200 mg·mL⁻¹ or extrapolating to predict the injection force for a composition having a protein concentration below 100 mg·mL⁻¹ or above 200 mg·mL⁻¹;
121) Use according to any of embodiments 116 to 120 in which the injection force of the composition is less than or equal to 90, 80, 70, 60, 50, 50, 45, 40, 35, 30, 25% of the predicted injection force.
122) Use according to any of embodiments 116 to 121 in which the composition has an injection force below 25 N, preferably below 20, 19, 18, 17, 16, 15, 14, 13, 12, 11, 10, 9, 8, 7, 6, 5, 4, 3, 2, 1 N.
123) Use according to any of embodiments 116 to 122 in which the injection force is measured by extruding the composition through a 27 G ½″ needle at 2 or 4 mL/minute
124) Use of albumin to control or reduce the viscosity of a composition comprising antibodies.
125) Use according to embodiment 124 which the composition has a viscosity no more than 100% of the viscosity of an equivalent composition in which the albumin component is replaced with antibody.
126) Use according to embodiment 124 or 125 in which the viscosity of the composition is less than or equal to 90, 80, 70, 60, 50, 50, 45, 40, 35, 30, 25% of viscosity of an equivalent composition which does not include albumin.
127) Use according to any of embodiments 124 to 126 in which the viscosity of the composition is no more than 100% of the predicted by preparing antibody compositions of about 100, about 150 and about 200 mg·mL⁻¹, calculating a line, or curve, of best fit and predicting the viscosity for a composition having a protein concentration between 100 and 200 mg·mL⁻¹ or extrapolating to predict the viscosity for a composition having a protein concentration below 100 mg·mL⁻¹ or above 200 mg·mL⁻¹.
128) Use according to any of embodiments 124 to 127 which the viscosity of the composition is less than or equal to 90, 80, 70, 60, 50, 50, 45, 40, 35, 30, or 25% of the predicted viscosity.
129) Use according to any of embodiments 124 to 128 which the composition has a viscosity below 500 mPa·s, preferably below 450, 400, 350, 300, 250, 200, 150, 100, 50, 40, 30, or 20 mPa·s.
130) Use according to any of embodiments 124 to 129 in which the viscosity of the composition is measured by rheometry (e.g. Anton Paar rheometer MCR 301) at 25° C., shear rate 10-10000 its using CP25-1 geometry.
131) The use according to any of embodiments 114 to 130 in which, prior to stabilization, the antibody is in a lyophilized state.
132) The use according to any of embodiments 114 to 131 in which, prior to stabilization, the antibody is in a liquid state.
133) The antibody preparation according to any of embodiments 1 to 104 or obtained by the method of any of embodiments 105 to 113 for treatment or prevention of disease or of a medical condition.
134) The antibody preparation (composition, e.g. liquid composition) according to embodiment 133 in which the antibody is Bevacizumab and the disease or medical condition is selected from the group consisting of: Metastatic colorectal cancer; advanced and/or metastatic renal cell cancer; advanced, metastatic or recurrent non-squamous Non-Small Cell Lung Cancer; metastatic breast cancer; relapsed high grade malignant glioma; or epithelial ovarian, fallopian tube or primary peritoneal cancer.
135) The antibody preparation (composition, e.g. liquid composition) according to embodiment 134 further comprising one or more other pharmacologically active compounds such as:
   a) a chemotherapy agent such as:
      i) a fluoropyrimidine medicine (e.g. for treatment of advanced cancer in the large bowel e.g. in the colon or rectum);
      ii) paclitaxel or capecitabine (e.g. for treatment of breast cancer such as metastatic breast cancer);
      iii) a chemotherapy regimen containing platinum (e.g. for treatment of advanced non-small cell lung cancer).
   b) interferon (e.g. for treatment of kidney cancer such as advanced kidney cancer).
   c) carboplatin and/or paclitaxel (e.g. for treatment of advanced epithelial ovarian, fallopian tube, or primary peritoneal cancer).
   d) carboplatin and gemcitabine (e.g. for treatment of those patients whose disease has come back at least 6 months after the last time they were treated with a chemotherapy regimen containing a platinum).
136) The antibody preparation (composition, e.g. liquid composition) according to embodiment 133 in which the antibody is Rituximab and the disease or medical condition is selected from the group consisting of: Non-Hodgkin's lymphoma (NHL), Chronic lymphocytic leukemia (CLL), Rheumatoid arthritis, Granulomatosis with polyangiitis and Microscopic polyangiitis.
137) The antibody preparation (composition, e.g. liquid composition) according to embodiment 136 further comprising one or more other pharmacologically active compounds such as:
   a) chemotherapy agent (e.g. for treatment of Non-Hodgkin's Lymphoma e.g. stage III-IV follicular lymphoma)
   b) combination of chemotherapy agents e.g. cyclophosphamide, doxorubicin, vincristine, and prednisolone (e.g. for treatment of patients with CD20 positive diffuse large B cell non-Hodgkin's lymphoma)
   c) glucocorticoids (e.g. for induction of remission in adult patients with severe, active Granulomatosis with polyangiitis (Wegener's) (GPA) and Microscopic polyangiitis (MPA)).
138) The composition (e.g. liquid composition) according to any preceding embodiment in which the antibody comprises about 50 to about 250 mg·mL⁻¹ antibody and about 25 to about 75 mg·mL⁻¹ albumin.
139) The composition (e.g. liquid composition) according to embodiment 138 in which the antibody comprises Bevacizumab.
140) The composition (e.g. liquid composition) according to embodiment 139, in which the composition comprises about 60 mg·mL⁻¹ α,α-trehalose dehydrate, about 0.04% polysorbate 20, and about 0.9% NaCl and is about pH 6.2.
141) The composition (e.g. liquid composition) according to embodiment 138 in which the antibody comprises Rituximab.
142) The composition (e.g. liquid composition) according to embodiment 141, in which the composition comprises about 7.35 mg·mL⁻¹ sodium citrate, about 0.7 mg·mL⁻¹ polysorbate 80 and about 0.9 mg·mL⁻¹ sodium chloride and is about pH 6.5.
143) The composition according to any preceding embodiment in which the color of the composition is from clear and colorless to slightly yellowish
144) A container holding an antibody preparation (composition, e.g. liquid composition) according to any of embodiments 1 to 105 or 133 to 143 or obtained by the method of any of embodiments 105 to 113.
145) The container according to embodiment 143 in which the container is selected from the group consisting of: a syringe, vial or bottle.
146) A kit comprising a container according to embodiment 144 or 145.
147) The kit according to embodiment 146 comprising administration and/or dosage instructions.

The invention is further described by the following examples that should not be construed as limiting the scope of the invention.

### EXAMPLES

### Media and Solutions

Albumin: recombinant human serum albumin (HSA) (Recombumin® Prime, Novozymes Biopharma).

Antibody: Bevacizumab (Avastin®, Roche): 25 mg/mL in buffer (100 mg Bevacizumab, 240 mg α,α-trehalose dihydrate, 23.2 mg sodium phosphate (monobasic, monohydrate), 4.8 mg sodium phosphate (dibasic, anhydrous), 1.6 mg polysorbate 20, and water for injection (USP) to make a volume of 4 mL).

### Example 1

### Albumin Prevents Aggregation (Dimerization) of Composition Comprising 150 mg/mL IgG

A 200 μL (microliter) composition comprising 150 mg/mL IgG (Bevacizumab) and 50 mg/mL albumin (Recombumin® Prime) was prepared by mixing 102 μL (microliter) 293 mg/mL Bevacizumab, 50 μL (microliter) 200 mg/mL mL Recombumin® Prime, 2 μL (microliter) 1% Tween® 80, 17 μL (microliter) 20 mg/mL arginine, 27 μL (microliter) 489 mg/mL trehalose and 2 μL (microliter) water. The 293 mg/mL Bevacizumab was prepared by protein A sepharose column purification of Avastin®, followed by overnight dialysis in Slide-A-Lyzer dialysis cassettes against MilliQ water. The purified Bevacizumab solution was lyophilized and subsequently resuspended in MilliQ water to a concentration of 293 mg/mL. The resuspension was carried out by gentle rotation of the tube containing Bevacizumab powder and water at room temperature over several days. The composition, and a reference, was incubated in 50 μL plastic Eppendorf tubes sealed with parafilm at 40° C. for 29 days. The reference was Avastin® (Bevacizumab without albumin). Subsequently, the composition and reference were diluted with water to a Bevacizumab concentration of 1.5 mg/mL and analyzed by AF4 separation coupled to UV₂₈₀ₙₘ detection to determine the extent of antibody aggregation. AF4 separation was carried out using an Eclipse instrument (Wyatt Technology Europe GmbH) equipped with a short channel, 350S spacer and a 10 kDa regenerated cellulose membrane. The eluent was composed of 25 mM TRIS, 150 mM NaCl, 0.05% NaN₃, pH 7. Sample injection volume was 2 μL, and separation was achieved by using a channel flow of 1 mL/min and a cross flow gradient of 3-1 mL/min over 15 minutes. Quantification of Bevacizumab species was based on UV absorption at 280 nm (Dionex UltiMate 3000 VWD detector). Identification of eluted molecules was based on molecular weights, which were determined from UV absorption and multi angle light scattering (MALS detector and Astra software from Wyatt Technology Europe GmbH).

Table 1 shows that inclusion of albumin stabilizes the antibody as demonstrated by the reduction in dimer content.

TABLE 1

Antibody content

| Composition | Monomer | Dimer |
|---|---|---|
| Bevacizumab (Avastin ®) | 93.6% | 6.4% |
| Bevacizumab + albumin | 97.6% | 2.4% |

Reduction in dimer content indicates increased stability. Reduction in dimer content is desirable because it may reduce the likelihood of immunogenic reactions, may improve antibody activity, may improve dosing precision, and may improve the pharmacokinetic properties of the antibody. Consequently, reduction in dimer content may improve safety and efficacy of the antibody composition. However, reduction in dimer content was not observed in later performed experiments, the reason for this is unclear.

### Example 2

### Inclusion of Albumin in an Antibody Composition does not Adversely Affect the Viscosity of the Composition

The compositions in Table 2 were prepared and the injection force of each composition was compared. Composition 1 is Avastin buffer (51 mM sodium phosphate, pH 6.2, 60 mg/mL α,α-trehalose dehydrate, 0.04% polysorbate 20). Compositions 3 to 6 were prepared in Avastin buffer. Composition 2 was 50 mg/mL albumin diluted in buffer (145 mM NaCl, 8 mM octanoate, 50 mg/L polysorbate 80).

The injection force was measured by texture analysis using a TA.XT.plus Texture Analyser (Stable Micro Systems, Godalming, UK) at an injection speed of 2 mL/min using a 1-mL luer lock syringe equipped with a 30 G ½″ clinical needle.

TABLE 2

| Composition | Bevacizumab (mg/mL) | Albumin (mg/mL) |
|---|---|---|
| 1 | 0 | 0 |
| 2 | 0 | 50 |
| 3 | 110 | 0 |
| 4 | 100 | 50 |
| 5 | 183 | 0 |
| 6 | 200 | 50 |

The injection force of Avastin buffer (Composition 1) was comparable to that of albumin (Composition 2). Addition of 50 mg/mL albumin to a 100 mg/mL antibody composition led to a minor increase in the injection force (Composition 3 and Composition 4). Addition of 50 mg/mL albumin to a 200 mg/mL antibody composition did not lead to an increase in the injection force (Composition 5 and Composition 6) and appeared to reduce the injection force.

A lower injection force is more desirable than a higher injection force, because it makes the composition easier to deliver and less painful to receive. Therefore, a lower injection force may improve patient compliance.

### Example 3

### Inclusion of Albumin in a Bevacizumab Antibody Composition does not Adversely Affect the Viscosity of the Composition

The compositions in Table 3 were prepared. The injection force and viscosity of each composition was compared. All compositions were prepared in 60 mg/ml α,α-trehalose dihydrate, 0.04% polysorbate 20 and 0.9% NaCl, pH 6.2 (Avastin buffer)

The injection force was measured by texture analysis using a TA.XT.plus Texture Analyser (Stable Micro Systems, Godalming, UK) at an injection speed of 2 mL/min using a 1-mL luer lock syringe equipped with a 27 G ½″ clinical needle.

The viscosity was measured by Anton Paar rheometer MCR 301 at 25° C., shear rate 10-10000 1/s using CP25-1 geometry.

The following relationship between injection force and Bevacizumab concentration between 100-200 mg/mL was found: Y=0.1966e^{0.0183X}, where Y is the injection force and X is the concentration of Bevacizumab. (3 data points, R²=98.1%)

The following relationship between viscosity and Bevacizumab concentration between 100-400 mg/mL was initially calculated as Y=1.083e^{0.0224X}, where Y is the viscosity, and X is the concentration. (4 data points, R²=99.5%), this was subsequently found to have been calculated on an inappropriate data set and therefore re-calculated and corrected to Y=0.6992e^{0.0241X} (3 data points, R²=97.2%),

These equations were used to calculate the expected injection force and viscosity of compositions containing total protein concentrations of about 150 mg·mL⁻¹ (Composition 4), about 200 mg·mL⁻¹ (Composition 6) and about 250 mg·mL⁻¹ (Composition 8).

TABLE 3

| | | | | | | | Expected |
|---|---|---|---|---|---|---|---|
| | | | | | | Expected | Viscosity |
| | | | | | | Viscosity | (mPa * s) |
| | | | | Expected | | (mPa * s) | (corrected |
| | Bevacizumab | Albumin | Injection | Injection | Viscosity | (using Y = | using Y = |
| Composition | (mg/mL) | (mg/mL) | Force (N) | Force (N) | (mPa * s) | 1.083e^{0.0224X}) | 0.6992e^{0.0241X}) |
| 1 | 0 | 0 | 0.86 | — | 3.0 | — | |
| 2 | 0 | 53 | 0.86 | — | 4.3 | — | |
| 3 | 97 | 0 | 1.32 | — | 8.8 | — | |
| 4 | 97 | 50 | 1.12 | 3 | 7.8 | 31 | 26 |
| 5 | 154 | 0 | 2.65 | — | 20.5 | — | — |
| 6 | 154 | 40 | 3.41 | 8 | 24.7 | 96 | 87 |
| 7 | 202 | 0 | 8.24 | — | 97.7 | — | — |
| 8 | 202 | 56 | 11.66 | 19 | 278.3 | 293 | 289 |

The injection force of Avastin buffer (Composition 1) was comparable to that of about 50 mg/mL albumin (Composition 2). Addition of 50 mg/mL albumin to a 100 mg/mL antibody composition led to a minor decrease in the injection force and the viscosity was comparable for compositions with and without 50 mg/mL albumin (Composition 3 and Composition 4). Addition of 50 mg/mL albumin to a 150 mg/mL antibody composition led to a minor increase in the injection force and the viscosity (Composition 5 and Composition 6). Addition of 50 mg/mL albumin to a 200 mg/mL antibody composition led to an increase in the injection force and an increase in the viscosity (Composition 7 and Composition 8). The results are surprising because the observed injection force was lower than the predicted injection force, and the observed viscosity was lower than the predicted viscosity.

A lower injection force is more desirable than a higher injection force, because it makes the composition easier to deliver and less painful to receive. Therefore, a lower injection force may improve patient compliance. A lower viscosity is desirable because it makes the composition easier to manufacture.

### Example 4

### Inclusion of Albumin in a Rituximab Antibody Composition does not Adversely Affect the Viscosity of the Composition

The compositions in Table 4 were prepared. The injection force and viscosity of each composition was compared. All compositions were prepared in 7.35 mg/mL sodium citrate, 0.7 mg/mL polysorbate 80 and 9.0 mg/mL sodium chloride, pH 6.5 (Mabthera buffer)

The injection force was measured by texture analysis using a TA.XT.plus Texture Analyser (Stable Micro Systems, Godalming, UK) at an injection speed of 2 mL/min using a 1-mL luer lock syringe equipped with a 27 G ½″ clinical needle.

The viscosity was measured by Anton Paar rheometer MCR 301 at 25° C., shear rate 10-10000 1/s using CP25-1 geometry.

The following relationship between injection force and Rituximab concentration between 100-200 mg/mL was found: Y=0.1185e^{0.0218X}, where Y is the injection force, and X is the concentration. (3 data points, R²=99.5%).

The following relationship between viscosity and Rituximab concentration between 100-200 mg/mL was found: Y=0.4588e^{0.0265X}, where Y is the viscosity, and X is the concentration. (3 data points, R²=99.9%).

These equations were used to calculate the expected injection force and viscosity of compositions containing total protein concentrations of about 150 mg·mL⁻¹ (Composition 4), about 200 mg·mL⁻¹ (Composition 6) and about 250 mg·mL⁻¹ (Composition 8).

TABLE 4

| | | | | Expected | | Expected |
|---|---|---|---|---|---|---|
| | Rituximab | Albumin | Injection | Injection | Viscosity | viscosity |
| Composition | (mg/mL) | (mg/mL) | Force (N) | Force (N) | (mPa * s) | (mPa * s) |
| 1 | 0 | 0 | 0.95 | — | 3.22 | — |
| 2 | 0 | 51 | 0.99 | — | 3.39 | — |
| 3 | 111 | 0 | 1.1 | — | 6.61 | — |
| 4 | 111 | 50 | 1.35 | 3 | 9.04 | 24 |
| 5 | 163 | 0 | 2.85 | — | 23.5 | — |
| 6 | 144 | 49 | 2.77 | 9 | 14.8 | 92 |
| 7 | 215 | 0 | 9.75 | — | 93.4 | — |
| 8 | 196 | 53 | 6.34 | 28 | 43.7 | 346 |

The injection force of Mabthera buffer (Composition 1) was comparable to that of about 50 mg/mL albumin (Composition 2). Addition of 50 mg/mL albumin to a 100 mg/mL antibody composition led to a minor increase in the injection force and the viscosity (Composition 3 and Composition 4). Addition of 50 mg/mL albumin to a 150 mg/mL antibody composition led to a minor decrease in the injection force and the viscosity (Composition 5 and Composition 6). The results are surprising because the observed injection force was lower than the predicted injection force, and the observed viscosity was lower than the predicted viscosity. Furthermore, surprisingly, addition of 50 mg/mL albumin to an about 150 or an about 200 mg/mL antibody composition led to a decrease in both injection force and viscosity (Composition 5 and Composition 6; Composition 7 and Composition 8).

A lower injection force is more desirable than a higher injection force, because it makes the composition easier to deliver and less painful to receive. Therefore, a lower injection force may improve patient compliance. A lower viscosity is desirable because it makes the composition easier to manufacture.

### Example 5

### Inclusion of Albumin in a Rituximab Antibody Composition Affects the Viscosity of the Composition Less than Expected

The compositions in Table 5 were prepared. The viscosity of each composition was compared. All compositions were prepared in 7.35 mg/mL sodium citrate, 0.7 mg/mL polysorbate 80 and 9.0 mg/mL sodium chloride, pH 6.5 (Mabthera buffer)

The viscosity was measured by Anton Paar rheometer MCR 301 at 25° C., shear rate 10-10000 1/s using CP25-1 geometry.

The relationship between viscosity and Rituximab concentration from Example 4 was used to calculate the expected viscosity of compositions containing total protein concentrations of about 200 mg·mL⁻¹ (Composition 1), about 225 mg·mL⁻¹ (Composition 2) and about 250 mg·mL⁻¹ (Composition 3).

TABLE 5

| | Rituximab | Albumin | Viscosity | Expected viscosity |
|---|---|---|---|---|
| Composition | (mg/mL) | (mg/mL) | (mPa*s) | (mPa*s) |
| 1 | 199 | 0 | 124 | 92 |
| 2 | 199 | 27 | 155 | 178 |
| 3 | 199 | 57 | 202 | 346 |

Addition of 25-50 mg/mL albumin to a 200 mg/mL antibody composition led to an increase in the viscosity that was less than expected (Compositions 2 and 3). The decrease in viscosity resulting from the addition of 50 mg/mL to an about 200 mg/mL antibody composition observed in Example 4 Composition 8 was not observed in Example 5 Composition 3. The results are nonetheless surprising because the observed viscosity was lower than the predicted viscosity in both Compositions 2 and 3.

A lower viscosity is desirable because it makes the composition easier to manufacture.

### Example 6

### Albumin Prevents Aggregation of Composition Comprising 150 mg/mL IgG

Four 400 μL (microliter) compositions were prepared as described in Table 7. Stock solutions of trehalose (131.49 mg/mL), NaCl (4.38 mg/mL), phosphate (40 mM), albumin (Recombumin® Prime 20% diluted with double processed tissue culture water (Sigma) to 10%), and IgG antibody Bevacizumab (7.66 mg/mL, “BVC”) or Rituximab (5.92 mg/mL, “RTX”) or control samples without antibody (“CTR”) were mixed in the mass ratio corresponding to Table 7. All stock solutions were pH adjusted to pH 6.4. The Bevacizumab and Rituximab stock solutions were prepared by Protein A sepharose column purification of Avastin® and MabThera®, respectively, followed by dialysis in dialysis tubing Spectra/Por, molecular weight cut-off (mwco): 12-14 kDa against 20 mM phosphate buffer, pH 6.4, and afterwards against milliQ water. Dialysis was ended, when pH of the phosphate buffer or conductivity of the milliQ water were stable. After dialysis the compositions were lyophilized (in a freeze-dryer Heto CD8, manufactured by Heto Lab Equipment A/S, according to the program given in Table 6) and subsequently resuspended in double processed tissue culture water (Sigma) to the concentrations given in Table 6.

TABLE 6
Lyophilization program

| Duration | Temperature | |
|---|---|---|
| (h) | (° C.) | Vacuum |
| 3 | −30 | — |
| 1.5 | −10 | — |
| 3 | −30 | — |
| 51 | −30 | On |
| 40 | 20 | — |
| 10 | 30 | — |
| 1 | 5 | — |

TABLE 7
All compositions also contained 65.8 mg/mL trehalose,
2.2 mg/mL NaCl, 20 mM phosphate, pH 6.4

| | | Antibody | Albumin |
|---|---|---|---|
| | | concentration | concentration |
| Composition | IgG antibody | (mg/mL) | (mg/mL) |
| 1. BVC + alb | Bevacizumab | 150 | 0 |
| 2. BVC | | 150 | 50 |
| 3. RTX + alb | Rituximab | 150 | 0 |
| 4. RTX | | 150 | 50 |
| 5. CTR + alb | — | — | 0 |
| 6. CTR | | — | 50 |

CTR: Control (i.e. no antibody present)

Each Composition except 5 and 6 was divided in an “a” and “b” sample. All samples including Composition 5 and 6 were incubated in tightly sealed HPLC vials with glass inserts at 40° C. for 4 weeks (Bevacizumab) or 9 weeks (Rituximab). After incubation the compositions were diluted 100-fold with 20 mM phosphate, 100 mg/mL NaCl, pH 6.4, to an antibody concentration of 1.5 mg/mL and analyzed on a Brightwell 4200 MFI system for particle counting and sizing measurements. Three consecutive analyses were performed on three volumes of 300 μL. Data processing included removal of air bubbles and silicone drops and counting of particles in the range of 2 to 100 μm.

Tables 8, 9, 10, 11 and 12 show that inclusion of albumin (“alb”) stabilizes the antibody as demonstrated by the reduction in numbers of particles larger than 2 μm, larger than 10 μm, larger than 25 μm, 2 to 10 μm, and 2 to 25 μm, respectively.

TABLE 8
Particles larger than 2 μm (number/mL). Mean average and standard deviation was
calculated from three consecutive analyses.

| | T0 | | T4w at 40° C. | |
|---|---|---|---|---|
| | | Standard | | Standard |
| Sample | Mean average | deviation | Mean average | deviation |
| 1a. BVC + alb | Not determined | Not determined | 28216 | 1207 |
| 1b. BVC + alb | 61448 | 1155 | 10353 | 451 |
| 2a. BVC | 98272 | 3932 | 68652 | 3498 |
| 2b. BVC | 98844 | 7969 | 61008 | 2712 |
| | T0 | | T9w at 40° C. | |
| | | Standard | | Standard |
| Sample | Mean average | deviation | Mean average | deviation |
| 3a. RTX + alb | 9512 | 449 | 8381 | 387 |
| 3b. RTX + alb | 13644 | 259 | 15455 | 209 |
| 4a. RTX | 38552 | 1520 | 83895 | 2094 |
| 4b. RTX | 45543 | 1130 | 49780 | 972 |

| | T0 | | T4w at 40° C. | | T9w at 40° C. | |
|---|---|---|---|---|---|---|
| | Mean | Standard | Mean | Standard | Mean | Standard |
| Sample | average | deviation | average | deviation | average | deviation |
| 5. CTR + alb | 276 | 172 | 249 | 57 | 592 | 266 |
| 6. CTR | 164 | 106 | 159 | 148 | 126 | 59 |

T0: zero time point;
T4w at 40° C.: time point 4 weeks, incubation at 40° C.;
T9w at 40° C.: time point 9 weeks, incubation at 40° C.

The data of Table 8 show that addition of albumin to antibody immediately reduces the number of particles present larger than 2 μm, and that antibody compositions comprising albumin contain much fewer particles larger than 2 μm following prolonged incubation at 40° C. than antibody compositions that do not contain albumin.

TABLE 9
Particles larger than 10 μm (number/mL). Mean average and standard deviation was
calculated from three consecutive analyses.

| | T0 | | T4w at 40° C. | |
|---|---|---|---|---|
| | | Standard | | Standard |
| Sample | Mean average | deviation | Mean average | deviation |
| 1a. BVC + alb | nd | nd | 2036 | 100 |
| 1b. BVC + alb | 5245 | 415 | 519 | 64 |
| 2a. BVC | 3508 | 4 | 2652 | 208 |
| 2b. BVC | 4325 | 476 | 2135 | 284 |
| | T0 | | T9w at 40° C. | |
| | | Standard | | Standard |
| Sample | Mean average | deviation | Mean average | deviation |
| 3a. RTX + alb | 420 | 36 | 308 | 17 |
| 3b. RTX + alb | 1426 | 121 | 603 | 69 |
| 4a. RTX | 1882 | 152 | 2677* | 134 |
| 4b. RTX | 2315 | 96 | 713 | 75 |

| | T0 | | T4w at 40° C. | | T9w at 40° C. | |
|---|---|---|---|---|---|---|
| | Mean | Standard | Mean | Standard | Mean | Standard |
| | average | deviation | average | deviation | average | deviation |
| 5. CTR + alb | 23 | 12 | 26 | 15 | 25 | 6 |
| 6. CTR | 5 | 7 | 3 | 3 | 15 | 4 |

T0: zero time point;
T4w at 40° C.: time point 4 weeks, incubation at 40° C.;
T9w at 40° C.: time point 9 weeks, incubation at 40° C.

The data of Table 9 show that addition of albumin to antibody slightly increases the number of particles larger than 10 μm present and that, when the data are averaged, antibody compositions comprising albumin contain much fewer particles larger than 10 μm following prolonged incubation at 40° C. than antibody compositions that do not contain albumin. It is expected that the 9 week time point for Sample 4a is an outlier and that the representative average is around 700 (sample 4b).

TABLE 10
Particles larger than 25 μm (number/mL). Mean average and standard deviation was
calculated from three consecutive analyses.

| | T0 | | T4w at 40° C. | |
|---|---|---|---|---|
| | | Standard | | Standard |
| Sample | Mean average | deviation | Mean average | deviation |
| 1a. BVC + alb | nd | nd | 188 | 26 |
| 1b. BVC + alb | 322 | 32 | 27 | 9 |
| 2a. BVC | 197 | 28 | 147 | 41 |
| 2b. BVC | 268 | 25 | 142 | 28 |
| | T0 | | T9w at 40° C. | |
| | | Standard | | Standard |
| Sample | Mean average | deviation | Mean average | deviation |
| 3a. RTX + alb | 32 | 8 | 10 | 3 |
| 3b. RTX + alb | 160 | 14 | 40 | 10 |
| 4a. RTX | 62 | 20 | 72 | 20 |
| 4b. RTX | 63 | 6 | 22 | 11 |

| | T0 | | T4w at 40° C. | | T9w at 40° C. | |
|---|---|---|---|---|---|---|
| | Mean | Standard | Mean | Standard | Mean | Standard |
| | average | deviation | average | deviation | average | deviation |
| 5. CTR + alb | 3 | 6 | 7 | 6 | 5 | 2 |
| 6. CTR | 1 | 2 | 1 | 2 | 1 | 2 |

T0: zero time point;
T4w at 40° C.: time point 4 weeks, incubation at 40° C.;
T9w at 40° C.: time point 9 weeks, incubation at 40° C.

The number of particles (sized larger than 25 micrometers) observed is insignificant and outside the normal analytical window of the particle counting instrument.

TABLE 11
Particles from 2 to 10 μm (number/mL). Mean average was
calculated from the difference between number of particles
larger than 10 μm and number of particles larger than 2 μm.

| Sample | T0 | T4 w at 40° C. |
|---|---|---|
| 1a. BVC + alb | nd | 26180 |
| 1b. BVC + alb | 56203 | 9834 |
| 2a. BVC | 94764 | 65999 |
| 2b. BVC | 94519 | 58873 |
| | T0 | T9 w at 40° C. |
| 3a. RTX + alb | 9092 | 8073 |
| 3b. RTX + alb | 12218 | 14852 |
| 4a. RTX | 36670 | 81217 |
| 4b. RTX | 43227 | 49067 |

| | T0 | T4 w at 40° C. | T9 w at 40° C. |
|---|---|---|---|
| 5. CTR + alb | 253 | 223 | 567 |
| 6. CTR | 159 | 155 | 111 |

T0: zero time point.
T4 w at 40° C.; time point 4 weeks, incubation at 40° C.;
T9 w at 40° C.: time point 9 weeks, incubation at 40° C.

The data of Table 11 show that addition of albumin to antibody immediately reduces the number of 2 to 10 μm particles present, and that antibody compositions comprising albumin contain much fewer 2 to 10 μm particles following prolonged incubation at 40° C. than antibody compositions that do not contain albumin.

TABLE 12
Particles from 2 to 25 μm (number/mL). Mean average was
calculated from the difference between number of particles
larger than 25 μm and number of particles larger than 2 μm.

| Sample | T0 | T4 w at 40° C. |
|---|---|---|
| 1a. BVC + alb | nd | 28028 |
| 1b. BVC + alb | 61126 | 10326 |
| 2a. BVC | 98075 | 68505 |
| 2b. BVC | 98576 | 60866 |
| | T0 | T9 w at 40° C. |
| 3a. RTX + alb | 9480 | 8371 |
| 3b. RTX + alb | 13484 | 15415 |
| 4a. RTX | 38490 | 83823 |
| 4b. RTX | 45480 | 49758 |

| | T0 | T4 w at 40° C. | T9 w at 40° C. |
|---|---|---|---|
| 5. CTR + alb | 273 | 242 | 588 |
| 6. CTR | 163 | 157 | 125 |

T0: zero time point;
T4 w at 40° C.: time point 4 weeks, incubation at 40° C.;
T9 w at 40° C.: time point 9 weeks, incubation at 40° C.

The data of Table 12 show that addition of albumin to antibody immediately reduces the number of 2-25 μm particles present, and that antibody compositions comprising albumin contain much fewer 2-25 μm particles following prolonged incubation at 40° C. than antibody compositions that do not contain albumin.

The data of Table 13 shows that inclusion of albumin stabilizes the antibody as demonstrated by the percentage reduction in numbers of particles larger than 2 μm and 2 to 10 μm, respectively. The percentage reduction was based on mean averages of two subsamples a and b having the same composition with albumin and two subsamples a and b without albumin. There was some variation in particle content between subsamples (see Tables 8 and 11), but this variation was smaller than the calculated reduction in particle content.

TABLE 13
Percentage reduction in particle content in samples with albumin
relative to samples without albumin. Calculations were based on mean
averages of two subsamples a and b with the same composition.

| | Particle size | T0 | T4 w | T9 w |
|---|---|---|---|---|
| BVC | larger than 2 μm | 38% | 70% | nd |
| | 2 to 10 μm | 41% | 71% | nd |
| RTX | larger than 2 μm | 72% | nd | 82% |
| | 2 to 10 μm | 73% | nd | 82% |

nd: not determined;
T4 w: time point, 4 weeks;
T9 w: time point, 9 weeks (despite the observation that the 4 week data point for sample 4a is likely to be an outlier, both data points (4a and 4b) from Table 9 were used in these calculations).

Reduction in particle numbers indicates increased stability. Reduction in particle numbers is desirable because it may reduce the likelihood of immunogenic reactions, may improve antibody activity, may improve dosing precision, and may improve the pharmacokinetic properties of the antibody. Consequently, reduction in particle numbers may improve safety and efficacy of the antibody composition.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A liquid composition of antibody, the composition comprising antibodies at a concentration of greater than or equal to 140 mg/mL and albumin at a concentration of greater than or equal to 10 mg/mL.

2. The liquid composition according to claim 1, wherein the antibody concentration is from 140 to 200 mg/mL.

3. The liquid composition according to claim 1, which contains at least 1 percentage point fewer antibody aggregates, e.g. particulates, compared with a reference composition which is identical to the antibody composition with the exception that it does not comprise albumin, preferably at least 10 percentage point fewer antibody aggregates.

4. The liquid composition according to claim 3, in which the composition contains at least 1 percentage point fewer particulates (e.g. having size from 1 to 100 micrometers) following exposure to 40° C. for at least 28 days, such as 29 days or at least 63 days, preferably at least 10 percentage point fewer antibody aggregates.

5. The liquid composition according to claim 4, in which the composition contains at least 1 percentage point fewer particulates having size from 2 to 10 micrometers following exposure to 40° C. for at least 28 days, such as 29 days or at least 63 days.

6. The liquid composition according to claim 5, in which the composition contains at least 10 percentage point fewer particulates having size from 2 to 10 micrometers following exposure to 40° C. for at least 28 days, such as at least 29 days or at least 63 days.

7. The liquid composition according claim 1, which has an injection force no more than 100% of the injection force of an equivalent composition in which the albumin component is replaced with antibody.

8. The liquid composition according to claim 7 in which the injection force is less than or equal to 90% of the injection force of an equivalent composition in which the albumin component is replaced with antibody.

9. The liquid composition according to claim 1, in which the composition has an injection force below 15 N.

10. The liquid composition according to claim 1, which has a viscosity no more than 100% of the viscosity of an equivalent composition in which the albumin component is replaced with antibody.

11. The liquid composition according to claim 10 in which the viscosity is less than or equal to 90% of viscosity of an equivalent composition which does not include albumin.

12. The liquid composition according to claim 1, which has a viscosity below 300 mPa·s.

13. The liquid composition according to claim 1, in which the antibody is an IgG, preferably a monoclonal IgG.

14. The liquid composition according to claim 1, in which the albumin concentration is from about 40 to about 60 mg/mL.

15. The liquid composition according to claim 1, in which the albumin has at least 80% sequence identity to SEQ ID NO: 2, preferably consisting of SEQ ID NO: 2.

16. The liquid composition according to claim 1, which has a pH from about 6 to 7, a cation concentration of from about 10 to about 185 mM, an octanoate concentration of from about 0 to about 16 mM, and a detergent concentration of from about 0 to about 0.1 (w/v) %.

17. A method for producing a composition of antibody, the method comprising combining antibodies with albumin to produce a composition having an antibody concentration of greater than or equal to about 140 mg/mL and an albumin concentration of greater than or equal to about 10 mg/mL.

18. (canceled)

19. The method according to claim 17, in which, prior to the combining step, the antibody is in a liquid state.

20. The method according to claim 17, in which, prior to the combining step, the antibody is in a lyophilized state.

21. The method according to claim 17, comprising lyophilizing the resultant composition.

22. The composition according to claim 1, in which the antibody content is at least 95% monomer, preferably at least 97% monomer.

23. A method to stabilize an antibody composition in which antibody is present at a concentration of greater than or equal to about 140 mg/mL, the method comprising adding albumin to the antibody composition such that the antibody composition is stabilized.

24. A method to control or reduce the injection force of a composition comprising antibodies, the method comprising adding albumin to the composition comprising antibodies such that the injection force is controlled or reduced.

25. A method to control or reduce the viscosity of a composition comprising antibodies, the method comprising adding albumin to the composition comprising antibodies such that the viscosity is reduced.

26. (canceled)
